(19) United States
(12) Patent Application Publication
(10) Pub. No.: US 2010/0279884 A1
(43) Pub. Date: Nov. 4, 2010

(54) PRIMERS, PROBES, MICROARRAY, AND METHOD FOR SPECIFIC DETECTION OF NINE RESPIRATORY DISEASE-ASSOCIATED BACTERIAL SPECIES

(75) Inventors: Ji-young OH, Yongin-si (KR); Nam HUH, Yongin-si (KR); Sang-hyun PEAK, Yongin-si (KR); Jong-Suk CHUNG, Yongin-si (KR); Soon-min MA, Yongin-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/616,516

(22) Filed: Dec. 27, 2006

(30) Foreign Application Priority Data
Dec. 27, 2005 (KR) 10-2005-0130610

(51) Int. Cl. C40B 30/04; C07H 21/04; C40B 40/06; C12Q 1/68

(52) U.S. Cl. 506/9; 536/24.33; 506/16; 435/6; 536/24.32

(57) ABSTRACT

Provided herein are a primer set capable of amplifying target sequence(s) of nine respiratory disease-associated bacterial species, a probe set specifically hybridizing with the target sequence(s), a microarray comprising the probe set, and a method of detecting one or more of the nine respiratory disease-associated bacterial species using the probe set.

This application claims priority to Korean Patent Application No. 10-2005-0130610, filed Dec. 27, 2005, and all the benefits accruing therefrom under 35 U.S.C. §119, the disclosure of which is incorporated herein in its entirety by reference.

### BACKGROUND OF THE INVENTION

1. Field of the Invention

The present invention relates to a primer set for amplifying target sequence(s) of nine respiratory disease-associated bacterial species, a probe set specifically hybridizing with the target sequence(s) of the nine respiratory disease-associated bacterial species, a microarray comprising the probe set, and a method of detecting the nine respiratory disease-associated bacterial species using the probe set.

2. Description of the Related Art

Typically, two sufficiently complementary single-stranded nucleic acids can hybridize to form a double helical structure in which the two antiparallel nucleic acid chains are held together by hydrogen bonds between complementary bases under conditions that promote their hybridization. Under appropriate conditions, DNA/DNA, RNA/DNA, or RNA/RNA hybrids may be formed.

A “probe” is a single-stranded nucleic acid sequence that is complementary to some particular degree with a nucleic acid sequence (“target”) to be detected. When needed, a probe may be labeled. The use of nucleic acid hybridization as a procedure for the detection of particular nucleic acid sequences is disclosed in U.S. Pat. No. 4,851,330, and U.S. Pat. No. 5,288,611.

Broadly, there are two fundamental nucleic acid hybridization procedures. In one procedure, known as “in-solution” hybridization, both a “probe” nucleic acid and a “target” nucleic acid in a test sample are free in solution. In the other procedure, one nucleic acid is immobilized in or on a solid substrate and the second nucleic acid is free in solution. For example, the position of a target nucleic acid present in a gel after electrophoresis may be located by using a solution of labeled probe nucleic acid that can hybridize with the target nucleic acid in the gel under appropriate conditions.

Probes for the detection of a few respiratory disease-associated bacteria are currently known. For example, U.S. Pat. No. 5,830,654 discloses hybridization assay probes for Haemophilus influenzae comprising oligonucleotides of about 14-18 nucleotides. U.S. Pat. No. 5,525,718 discloses oligonucleotides selectively hybridizing with a specific gene (e.g., the entE gene) of Staphylococcus aureus. U.S. Pat. No. 6,001,564 discloses primers or probes specific to Escherichia coli, Klebsiella pneumoniae, Pseudomonas aeruginosa, Proteus mirabilis, Streptococcus pneumoniae, Staphylococcus aureus, Staphylococcus epidermis, Haemophilus influenzae, and Moraxella catarrhalis.

However, no primer set capable of amplifying target sequences commonly found in the 23S rRNA genes of nine bacterial species (Chlamydophila pneumoniae, Haemophilus influenza, Klebsiella pneumoniae, Legionella pneumophila, Moraxella catarrhalis, Mycoplasma pneumoniae, Pseudomonas aeruginosa, Staphylococcus aureus, and Streptococcus pneumoniae) known to be associated with respiratory disease is reported. Furthermore, no probe(s) specific to the target sequence(s) of the 23S rRNA genes of the nine bacterial species is reported. The probes and primers are used in a specific and selective method for detecting the presence or absence of a bacterial species associated with respiratory disease.

### SUMMARY OF THE INVENTION

The present invention provides a primer set for amplifying target sequence(s) of nine respiratory disease-associated bacterial species.

The oligonucleotide primer set comprises at least one oligonucleotide set selected from the group consisting of: an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2; an oligonucleotide set comprising an oligonucleotide consisting of at least 10 contiguous nucleotides of SEQ ID NO: 3 and an oligonucleotide consisting of at least 10 contiguous nucleotides of SEQ ID NO: 4; an oligonucleotide set comprising an oligonucleotide consisting of at least 10 contiguous nucleotides of SEQ ID NO: 5 and an oligonucleotide consisting of at least 10 contiguous nucleotides of a sequence selected from the group consisting of SEQ ID NO: 6 and SEQ ID NO:7; and an oligonucleotide set comprising an oligonucleotide consisting of at least 10 contiguous nucleotides of SEQ ID NO: 8 and an oligonucleotide consisting of at least 10 contiguous nucleotides of SEQ ID NO: 9.

The present invention also provides a probe set for detecting one or more of nine respiratory disease-associated bacterial species. The probe set is specific to target sequence(s) amplified by the primer set.

The oligonucleotide probe set comprises at least one oligonucleotide probe selected from the group consisting of an oligonucleotide probe comprising an oligonucleotide consisting of at least 10 contiguous nucleotides of SEQ ID NO: 10 or a complement of the oligonucleotide; an oligonucleotide probe comprising an oligonucleotide consisting of at least 10 contiguous nucleotides of a sequence selected from the group consisting of SEQ ID NOS: 11-14 or a complement of the oligonucleotide; an oligonucleotide probe comprising an oligonucleotide consisting of at least 10 contiguous nucleotides of SEQ ID NO: 15 or a complement of the oligonucleotide; an oligonucleotide probe comprising an oligonucleotide consisting of at least 10 contiguous nucleotides of a sequence selected from the group consisting of SEQ ID NOS: 16-18 or a complement of the oligonucleotide; an oligonucleotide probe comprising an oligonucleotide consisting of at least 10 contiguous nucleotides of SEQ ID NO: 19 or a complement of the oligonucleotide; an oligonucleotide probe comprising an oligonucleotide consisting of at least 10 contiguous nucleotides of SEQ ID NO: 20 or a complement of the oligonucleotide; an oligonucleotide probe comprising an oligonucleotide consisting of at least 10 contiguous nucleotides of a sequence selected from the group consisting of SEQ ID NOS: 21-23 or a complement of the oligonucleotide; an oligonucleotide probe comprising an oligonucleotide consisting of at least 10 contiguous nucleotides of a sequence selected from the group consisting of SEQ ID NOS: 24-26 or a complement of the oligonucleotide; an oligonucleotide probe comprising an oligonucleotide consisting of at least 10 contiguous nucleotides of SEQ ID NO: 27 or a complement of the oligonucleotide; an oligonucleotide probe comprising an oligonucleotide consisting of at least 10 contiguous nucleotides of SEQ ID NO: 28 or a complement of the oligonucleotide; an oligonucleotide probe comprising an oligonucleotide consisting of at least 10 contiguous nucleotides of SEQ ID NO: 29 or a complement of the oligonucleotide; an oligonucleotide probe comprising an oligonucleotide consisting of at least 10 contiguous nucleotides of a sequence selected from the group consisting of SEQ ID NOS: 30-31 or a complement of the oligonucleotide; and an oligonucleotide probe comprising an oligonucleotide consisting of at least 10 contiguous nucleotides of a sequence selected from the group consisting of SEQ ID NOS: 32-35 or a complement of the oligonucleotide.

The present invention also provides a microarray comprising the probe set and a method of detecting one or more of the nine respiratory disease-associated bacterial species using the probe set.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram illustrating the positions of target sequences in the 23S rRNA gene; and

FIG. 2 shows electrophoretic results of polymerase chain reaction (“PCR”) products obtained by PCR using four primer sets of the present invention.

### DETAILED DESCRIPTION OF THE INVENTION

The present invention provides an oligonucleotide primer set for amplifying at least one target sequence of a 23S rRNA gene of at least one bacterial species selected from the group consisting of Chlamydophila pneumoniae, Haemophilus influenzae, Mycoplasma pneumoniae, Klebsiella pneumoniae, Legionella pneumophila, Moraxella catarrhalis, Pseudomonas aeruginosa, Staphylococcus aureus, and Streptococcus pneumoniae. The oligonucleotide primer set comprises at least one oligonucleotide set selected from the group consisting of: an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2; an oligonucleotide set comprising an oligonucleotide consisting of at least 10 contiguous nucleotides of SEQ ID NO: 3 and an oligonucleotide consisting of at least 10 contiguous nucleotides of SEQ ID NO: 4; an oligonucleotide set comprising an oligonucleotide consisting of at least 10 contiguous nucleotides of SEQ ID NO: 5 and an oligonucleotide consisting of at least 10 contiguous nucleotides of a sequence selected from the group consisting of SEQ ID NO: 6 and SEQ ID NO:7; and an oligonucleotide set comprising an oligonucleotide consisting of at least 10 contiguous nucleotides of SEQ ID NO: 8 and an oligonucleotide consisting of oat least 10 contiguous nucleotides of SEQ ID NO: 9.

The target sequence for the primer set of the invention can be at least one sequence selected from the group consisting of a nucleotide sequence corresponding to positions 124-365, a nucleotide sequence corresponding to positions 853-1353, a nucleotide sequence corresponding to positions 2483-2932, and a nucleotide sequence corresponding to positions 3041-3198 of bacterial 23s rRNA.

The primer set of the invention can be an oligonucleotide primer set for amplifying a nucleotide region corresponding to positions 124-365 of the 23S rRNA gene of at least one bacterial species selected from the group consisting of Chlamydophila pneumoniae, Haemophilus influenzae, Mycoplasma pneumoniae, Klebsiella pneumoniae, Legionella pneumophila, Moraxella catarrhalis, Pseudomonas aeruginosa, Staphylococcus aureus, and Streptococcus pneumoniae, comprising an oligonucleotide consisting of at least 10 contiguous nucleotides of SEQ ID NO: 1 and an oligonucleotide consisting of at least 10 contiguous nucleotides of SEQ ID NO: 2.

The primer set of the invention can be an oligonucleotide primer set for amplifying a nucleotide region corresponding to positions 853-1353 of the 23S rRNA gene of at least one bacterial species selected from the group consisting of Chlamydophila pneumoniae, Haemophilus influenzae, Mycoplasma pneumoniae, Klebsiella pneumoniae, Legionella pneumophila, Moraxella catarrhalis, Pseudomonas aeruginosa, Staphylococcus aureus, and Streptococcus pneumoniae, comprising an oligonucleotide consisting of at least 10 contiguous nucleotides of SEQ ID NO: 3 and an oligonucleotide consisting of at least 10 contiguous nucleotides of SEQ ID NO: 4.

The primer set of the invention can be an oligonucleotide primer set for amplifying a nucleotide region corresponding to positions 2483-2932 of the 23S rRNA gene of at least one bacterial species selected from the group consisting of Chlamydophila pneumoniae, Haemophilus influenzae, Mycoplasma pneumoniae, Klebsiella pneumoniae, Legionella pneumophila, Moraxella catarrhalis, Pseudomonas aeruginosa, Staphylococcus aureus, and Streptococcus pneumoniae, comprising an oligonucleotide set comprising an oligonucleotide consisting of at least 10 contiguous nucleotides of SEQ ID NO: 5 and an oligonucleotide consisting of at least 10 contiguous nucleotides of a sequence selected from the group consisting of SEQ ID NO: 6 and SEQ ID NO:7.

The primer set of the invention can be an oligonucleotide primer set for amplifying a nucleotide region corresponding to positions 3041-3198 of the 23S rRNA gene of at least one bacterial species selected from the group consisting of Chlamydophila pneumoniae, Haemophilus influenzae, Mycoplasma pneumoniae, Klebsiella pneumoniae, Legionella pneumophila, Moraxella catarrhalis, Pseudomonas aeruginosa, Staphylococcus aureus, and Streptococcus pneumoniae. In an embodiment, the oligonucleotide primer set comprises an oligonucleotide set comprising an oligonucleotide consisting of at least 10 contiguous nucleotides of SEQ ID NO: 8 and an oligonucleotide consisting of at least 10 contiguous nucleotides of SEQ ID NO: 9.

The primer set of the invention can be an oligonucleotide primer set for amplifying nucleotide regions corresponding to positions 124-365, 853-1353, 2483-2932, and 3041-3198 of the 23S rRNA gene of at least one bacterial species selected from the group consisting of Chlamydophila pneumoniae, Haemophilus influenzae, Mycoplasma pneumoniae, Klebsiella pneumoniae, Legionella pneumophila, Moraxella catarrhalis, Pseudomonas aeruginosa, Staphylococcus aureus, and Streptococcus pneumoniae. In one embodiment, the primer set comprises an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2; an oligonucleotide set comprising an oligonucleotide consisting of at least 10 contiguous nucleotides of SEQ ID NO: 3 and an oligonucleotide consisting of at least 10 contiguous nucleotides of SEQ ID NO: 4; an oligonucleotide set comprising an oligonucleotide consisting of at least 10 contiguous nucleotides of SEQ ID NO: 5 and an oligonucleotide consisting of at least 10 contiguous nucleotides of a sequence selected from the group consisting of SEQ ID NO: 6 and SEQ ID NO:7; and an oligonucleotide set comprising an oligonucleotide consisting of at least 10 contiguous nucleotides of SEQ ID NO: 8 and an oligonucleotide consisting of oat least 10 contiguous nucleotides of SEQ ID NO: 9. In another embodiment, the primer set comprises an oligonucleotide set comprising an oligonucleotide consisting of SEQ ID NO: 1 and an oligonucleotide consisting of SEQ ID NO: 2; an oligonucleotide set comprising an oligonucleotide consisting of SEQ ID NO: 3 and an oligonucleotide consisting of SEQ ID NO: 4; an oligonucleotide set comprising an oligonucleotide consisting of SEQ ID NO: 5 and an oligonucleotide consisting of SEQ ID NO: 6 or SEQ ID NO:7; and an oligonucleotide set comprising an oligonucleotide consisting of SEQ ID NO: 8 and an oligonucleotide consisting of SEQ ID NO: 9.

The primer set of the invention was designed from target regions common to the 23S rRNA genes of the nine respiratory disease-associated bacterial species. The nine respiratory disease-associated bacterial species are Chlamydophila pneumoniae, Haemophilus influenza, Klebsiella pneumoniae, Legionella pneumophila, Moraxella catarrhalis, Mycoplasma pneumoniae, Pseudomonas aeruginosa, Staphylococcus aureus, and Streptococcus pneumoniae.

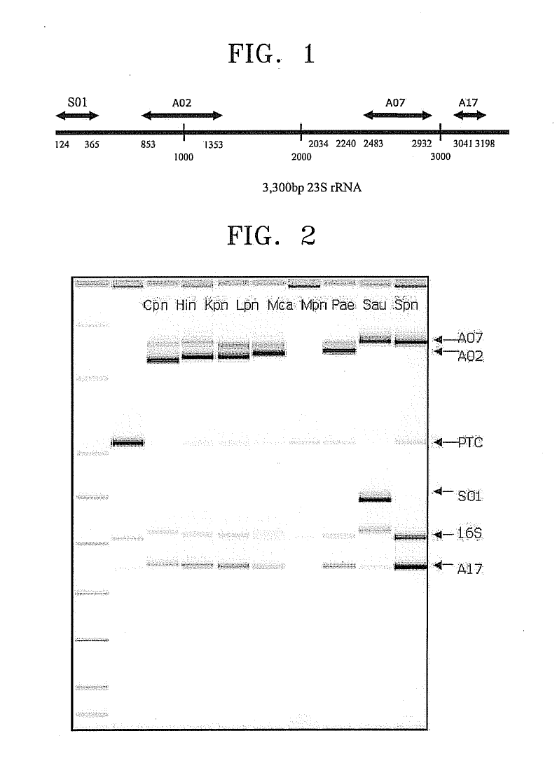

When performing PCR using the primer set of the present invention, a target sequence region sought to be amplified is selected from the nucleotide region corresponding to nucleotide positions 124-365 (represented by S01) of the 23S rRNA gene, the nucleotide region corresponding to nucleotide positions 853-1353 (represented by A02) of the 23S rRNA gene, the nucleotide region corresponding to nucleotide positions 2483-2932 (represented by A07) of the 23S rRNA gene, and the nucleotide region corresponding to nucleotide positions 3041-3198 (represented by A17) of the 23S rRNA gene. FIG. 1 is a diagram illustrating the nucleotide positions of the target sequences in the 23S rRNA gene.

The primer set of the invention was designed from the four target sequences common to the 23S rRNA genes of the nine respiratory disease-associated bacterial species. Exemplary examples of the primer set according to the present invention are presented in Table 1 below.

TABLE 1
Examples of primer set of the present invention

| Primer name | SEQ ID NO: | Remark |
|---|---|---|
| S01 | 1 | Forward primer for S01 amplification |
| S01 | 2 | Reverse primer for S01 amplification |
| A02 | 3 | Forward primer for A02 amplification |
| A02 | 4 | Reverse primer for A02 amplification |
| A07 | 5 | Forward primer for A07 amplification |
| A07 | 6 | Reverse primer for A07 amplification |
| A07 | 7 | Reverse primer for A07 amplification |
| A17 | 8 | Forward primer for A17 amplification |
| A17 | 9 | Reverse primer for A17 amplification |

The invention also provides an oligonucleotide probe set capable of hybridizing with at least one target sequence selected from the group consisting of nucleotide regions corresponding to positions 124-365, 853-1353, 2483-2932, and 3041-3198 of the 23S rRNA gene of at least one bacterial species selected from the group consisting of Chlamydophila pneumoniae, Haemophilus influenzae, Mycoplasma pneumoniae, Klebsiella pneumoniae, Legionella pneumophila, Moraxella catarrhalis, Pseudomonas aeruginosa, Staphylococcus aureus, and Streptococcus pneumoniae. In one embodiment, the oligonucleotide probe set comprises at least one oligonucleotide probe selected from the group consisting of an oligonucleotide probe comprising an oligonucleotide consisting of at least 10 contiguous nucleotides of SEQ ID NO: 10 or a complement of the oligonucleotide; an oligonucleotide probe comprising an oligonucleotide consisting of at least 10 contiguous nucleotides of a sequence selected from the group consisting of SEQ ID NOS: 11-14 or a complement of the oligonucleotide; an oligonucleotide probe comprising an oligonucleotide consisting of at least 10 contiguous nucleotides of SEQ ID NO: 15 or a complement of the oligonucleotide; an oligonucleotide probe comprising an oligonucleotide consisting of at least 10 contiguous nucleotides of a sequence selected from the group consisting of SEQ ID NOS: 16-18 or a complement of the oligonucleotide; an oligonucleotide probe comprising an oligonucleotide consisting of at least 10 contiguous nucleotides of SEQ ID NO: 19 or a complement of the oligonucleotide; an oligonucleotide probe comprising an oligonucleotide consisting of at least 10 contiguous nucleotides of SEQ ID NO: 20 or a complement of the oligonucleotide; an oligonucleotide probe comprising an oligonucleotide consisting of at least 10 contiguous nucleotides of a sequence selected from the group consisting of SEQ ID NOS: 21-23 or a complement of the oligonucleotide; an oligonucleotide probe comprising an oligonucleotide consisting of at least 10 contiguous nucleotides of a sequence selected from the group consisting of SEQ ID NOS: 24-26 or a complement of the oligonucleotide; an oligonucleotide probe comprising an oligonucleotide consisting of at least 10 contiguous nucleotides of SEQ ID NO: 27 or a complement of the oligonucleotide; an oligonucleotide probe comprising an oligonucleotide consisting of at least 10 contiguous nucleotides of SEQ ID NO: 28 or a complement of the oligonucleotide; an oligonucleotide probe comprising an oligonucleotide consisting of at least 10 contiguous nucleotides of SEQ ID NO: 29 or a complement of the oligonucleotide; an oligonucleotide probe comprising an oligonucleotide consisting of at least 10 contiguous nucleotides of a sequence selected from the group consisting of SEQ ID NOS: 30-31 or a complement of the oligonucleotide; and an oligonucleotide probe comprising an oligonucleotide consisting of at least 10 contiguous nucleotides of a sequence selected from the group consisting of SEQ ID NOS: 32-35 or a complement of the oligonucleotide.

The probe set of the invention can be an oligonucleotide probe set capable of hybridizing with the nucleotide region corresponding to the positions 3041-3198 of the 23S rRNA gene of Chlamydophila pneumoniae, comprising an oligonucleotide consisting of at least 10 contiguous nucleotides of SEQ ID NO: 10 or a complement of the oligonucleotide.

The probe set of the invention can be an oligonucleotide probe set capable of hybridizing with at least one target sequence selected from the group consisting of the nucleotide regions corresponding to the positions 853-1353 and 2483-2932 of the 23S rRNA gene of Haemophilus influenza. In one embodiment, the oligonucleotide probe set comprises at least one oligonucleotide probe selected from the group consisting of: an oligonucleotide probe comprising an oligonucleotide consisting of at least 10 contiguous nucleotides of a sequence selected from the group consisting of SEQ ID NOS: 11-14 or the complement of the oligonucleotide; and an oligonucleotide probe comprising an oligonucleotide consisting of at least 10 contiguous nucleotides of SEQ ID NO: 15 or the complement of the oligonucleotide. In another embodiment, the oligonucleotide probe set comprises an oligonucleotide probe comprising an oligonucleotide consisting of at least 10 contiguous nucleotides of a sequence selected from the group consisting of SEQ ID NOS: 11-14 or the complement of the oligonucleotide; and an oligonucleotide probe comprising an oligonucleotide consisting of at least 10 contiguous nucleotides of SEQ ID NO: 15 or the complement of the oligonucleotide.

The probe set of the invention can be an oligonucleotide probe set capable of hybridizing with the nucleotide region corresponding to the positions 2483-2932 of the 23S rRNA gene of Klebsiella pneumoniae, comprising an oligonucleotide consisting of at least 10 contiguous nucleotides of a sequence selected from the group consisting of SEQ ID NOS: 16-18 or the complement of the oligonucleotide.

The probe set of the invention can be an oligonucleotide probe set capable of hybridizing with at least one target sequence selected from the group consisting of the nucleotide regions corresponding to the positions 853-1353 and 2483-2932 of the 23S rRNA gene of Legionella pneumophila. In one embodiment, the oligonucleotide probe set comprises at least one oligonucleotide probe selected from the group consisting of an oligonucleotide probe comprising an oligonucleotide consisting of at least 10 contiguous nucleotides of SEQ ID NO: 19 or the complement of the oligonucleotide; and an oligonucleotide probe comprising an oligonucleotide consisting of at least 10 contiguous nucleotides of SEQ ID NO: 20 or the complement of the oligonucleotide. In another embodiment, the oligonucleotide probe set comprises an oligonucleotide probe comprising an oligonucleotide consisting of at least 10 contiguous nucleotides of SEQ ID NO: 19 or the complement of the oligonucleotide; and an oligonucleotide probe comprising an oligonucleotide consisting of at least 10 contiguous nucleotides of SEQ ID NO: 20 or the complement of the oligonucleotide.

The probe set of the invention can be an oligonucleotide probe set capable of hybridizing with at least one target sequence selected from the group consisting of the nucleotide regions corresponding to the positions 853-1353 and 2483-2932 of the 23S rRNA gene of Moraxella catarrhalis. In an embodiment, the oligonucleotide probe set comprises at least one oligonucleotide probe selected from the group consisting of an oligonucleotide probe comprising an oligonucleotide consisting of at least 10 contiguous nucleotides of a sequence selected from the group consisting of SEQ ID NOS: 21-23 or the complement of the oligonucleotide; and an oligonucleotide probe comprising an oligonucleotide consisting of at least 10 contiguous nucleotides of a sequence selected from the group consisting of SEQ ID NOS: 24-26 or the complement of the oligonucleotide. In an embodiment, the oligonucleotide probe set comprises an oligonucleotide probe comprising an oligonucleotide consisting of at least 10 contiguous nucleotides of a sequence selected from the group consisting of SEQ ID NOS: 21-23 or the complement of the oligonucleotide; and an oligonucleotide probe comprising an oligonucleotide consisting of at least 10 contiguous nucleotides of a sequence selected from the group consisting of SEQ ID NOS: 24-26 or the complement of the oligonucleotide.

The probe set of the invention can be an oligonucleotide probe set capable of hybridizing with the nucleotide region corresponding to the positions 3041-3198 of the 23S rRNA gene of Mycoplasma pneumoniae, comprising an oligonucleotide consisting of at least 10 contiguous nucleotides of SEQ ID NO: 27 or the complement of the oligonucleotide.

The probe set of the invention can be an oligonucleotide probe set capable of hybridizing with at least one target sequence selected from the group consisting of the nucleotide regions corresponding to the positions 853-1353 and 2483-2932 of the 23S rRNA gene of Pseudomonas aeruginosa. In an embodiment, the oligonucleotide probe set comprises an oligonucleotide probe selected from the group consisting of an oligonucleotide probe comprising an oligonucleotide consisting of at least 10 contiguous nucleotides of SEQ ID NO: 28 or the complement of the oligonucleotide; and an oligonucleotide probe comprising an oligonucleotide consisting of at least 10 contiguous nucleotides of SEQ ID NO: 29 or the complement of the oligonucleotide. In an embodiment, the oligonucleotide probe set comprises an oligonucleotide probe comprising an oligonucleotide consisting of at least 10 contiguous nucleotides of SEQ ID NO: 28 or the complement of the oligonucleotide; and an oligonucleotide probe comprising an oligonucleotide consisting of at least 10 contiguous nucleotides of SEQ ID NO: 29 or the complement of the oligonucleotide.

The probe set of the invention can be an oligonucleotide probe set capable of hybridizing with the nucleotide region corresponding to the positions 124-365 of the 23S rRNA gene of Staphylococcus aureus, comprising an oligonucleotide consisting of at least 10 contiguous nucleotides of a sequence selected from the group consisting of SEQ ID NOS: 30 and 31 or the complement of the oligonucleotide.

The probe set of the invention can, be an oligonucleotide probe set capable of hybridizing with the nucleotide region corresponding to the positions 2483-2932 of the 23S rRNA gene of Streptococcus pneumoniae, comprising an oligonucleotide consisting of at least 10 contiguous nucleotides of a sequence selected from the group consisting of SEQ ID NOS: 32-35 or the complement of the oligonucleotide.

The probe set of the invention can be an oligonucleotide probe set capable of hybridizing with a target sequence selected from the group consisting of the nucleotide regions corresponding to the positions 124-365, 853-1353, 2483-2932, and 3041-3198 of the 23S rRNA gene of a bacterial species selected from the group consisting of Chlamydophila pneumoniae, Haemophilus influenza, Klebsiella pneumoniae, Legionella pneumophila, Moraxella catarrhalis, Mycoplasma pneumoniae, Pseudomonas aeruginosa, Staphylococcus aureus, and Streptococcus pneumoniae. In one embodiment, the oligonucleotide probe set comprises at least one oligonucleotide probe selected from the group consisting of: an oligonucleotide probe comprising an oligonucleotide consisting of SEQ ID NO: 10, or the complement thereof; an oligonucleotide probe comprising an oligonucleotide consisting of a sequence selected from the group consisting of SEQ ID NOS: 11-14, or the complement thereof; an oligonucleotide probe comprising an oligonucleotide consisting of SEQ ID NO: 15, or the complement thereof; an oligonucleotide probe comprising an oligonucleotide consisting of a sequence selected from the group consisting of SEQ ID NOS: 16-18, or the complement thereof; an oligonucleotide probe comprising an oligonucleotide consisting of SEQ ID NO: 19, or the complement thereof; an oligonucleotide probe comprising an oligonucleotide consisting of SEQ ID NO: 20, or the complement thereof; an oligonucleotide probe comprising an oligonucleotide consisting of a sequence selected from the group consisting of SEQ ID NOS: 21-23, or the complement thereof; an oligonucleotide probe comprising an oligonucleotide consisting of a sequence selected from the group consisting of SEQ ID NOS: 24-26, or the complement thereof; an oligonucleotide probe comprising an oligonucleotide consisting of SEQ ID NO: 27, or the complement thereof; an oligonucleotide probe comprising an oligonucleotide consisting of SEQ ID NO: 28, or the complement thereof; an oligonucleotide probe comprising an oligonucleotide consisting of SEQ ID NO: 29, or the complement thereof; an oligonucleotide probe comprising an oligonucleotide consisting of a sequence selected from the group consisting of SEQ ID NOS: 30 and 31, or the complement thereof; and an oligonucleotide probe comprising an oligonucleotide consisting of a sequence selected from the group consisting of SEQ ID NOS: 32-35, or the complement thereof. In another embodiment, the oligonucleotide probe set comprises an oligonucleotide probe comprising an oligonucleotide consisting of SEQ ID NO: 10, or the complement thereof; an oligonucleotide probe comprising an oligonucleotide consisting of a sequence selected from the group consisting of SEQ ID NOS: 11-14, or the complement thereof; an oligonucleotide probe comprising an oligonucleotide consisting of SEQ ID NO: 15, or the complement thereof; an oligonucleotide probe comprising an oligonucleotide consisting of a sequence selected from the group consisting of SEQ ID NOS: 16-18, or the complement thereof; an oligonucleotide probe comprising an oligonucleotide consisting of SEQ ID NO: 19, or the complement thereof; an oligonucleotide probe comprising an oligonucleotide consisting of SEQ ID NO: 20, or the complement thereof; an oligonucleotide probe comprising an oligonucleotide consisting of a sequence selected from the group consisting of SEQ ID NOS: 21-23, or the complement thereof; an oligonucleotide probe comprising an oligonucleotide consisting of a sequence selected from the group consisting of SEQ ID NOS: 24-26, or the complement thereof; an oligonucleotide probe comprising an oligonucleotide consisting of SEQ ID NO: 27, or the complement thereof; an oligonucleotide probe comprising an oligonucleotide consisting of SEQ ID NO: 28, or the complement thereof; an oligonucleotide probe comprising an oligonucleotide consisting of SEQ ID NO: 29, or the complement thereof; an oligonucleotide probe comprising an oligonucleotide consisting of a sequence selected from the group consisting of SEQ ID NOS: 30 and 31, or the complement thereof; and an oligonucleotide probe comprising an oligonucleotide consisting of a sequence selected from the group consisting of SEQ ID NOS: 32-35, or the complement thereof.

The probe set of the invention specifically binds with PCR products amplified from target regions S01, A02, A07, and A17 of the 23S rRNA genes of the nine bacterial species obtained by PCR using the primer set of the invention. Thus, the probe set of the invention can be used to identify the nine bacterial species. The probe set of the present invention was designed by comparing the target regions S01, A02, A07, and A17 of the 23S rRNA genes of the nine bacterial species and selecting sequence(s) specifically present in each bacterial species.

As used herein, the term “probe” refers to a single-stranded nucleic acid sequence that can be base-paired with a complementary single-stranded target sequence to form a double-stranded molecule (hybrid).

As used herein, the term “hybridization” refers to the hydrogen bonding between two complementary strands of nucleic acid to form a double-stranded molecule (hybrid).

As used herein, “stringency” is the term used to describe a temperature and a solvent composition during hybridization and the subsequent processes. Under high stringency conditions, only highly complementary nucleic acid hybrids will be formed. Accordingly, the stringency of the hybridization assay conditions determines the amount of complementarity which should exist between two nucleic acid strands (probe and target) to form a hybrid. An example of a high stringency condition is a 0.12M phosphate buffer including equal moles of Na₂HPO₄ and NaH₂PO₄, 1 mM EDTA, and 0.02% sodium dodecylsulfate at 65° C. Stringency is chosen to maximize the difference in stability between probe-target hybrids and probe-non-target hybrids. The present invention also provides a microarray comprising a substrate, wherein the oligonucleotide probe set according to the invention is immobilized thereon.

As used herein, the term “microarray” refers to a high-density array of two or more groups of polynucleotides immobilized on a substrate. Here, each of the two or more groups of the polynucleotides is immobilized in a different predetermined region of the substrate. Microarrays are well known in the art. Examples of such microarrays are disclosed in U.S. Pat. No. 5,445,934 and U.S. Pat. No. 5,744,305 the disclosures of which are incorporated herein in their entireties by reference.

A method of detecting a respiratory disease-associated bacterial species selected from the group consisting of Chlamydophila pneumoniae, Haemophilus influenza, Klebsiella pneumoniae, Legionella pneumophila, Moraxella catarrhalis, Mycoplasma pneumoniae, Pseudomonas aeruginosa, Staphylococcus aureus, and Streptococcus pneumoniae is also provided. The method comprises contacting a sample to the oligonucleotide probe set according to the invention so that an oligonucleotide probe can hybridize with a target sequence present in the sample; and detecting a degree of hybridization between the oligonucleotide probe and the target sequence.

In an embodiment of the method, the sample comprises a PCR product. The PCR product can be obtained by PCR using template nucleic acid obtained from a bacterial species selected from the group consisting of Chlamydophila pneumoniae, Haemophilus influenza, Klebsiella pneumoniae, Legionella pneumophila, Moraxella catarrhalis, Mycoplasma pneumoniae, Pseudomonas aeruginosa, Staphylococcus aureus, and Streptococcus pneumoniae, and an oligonucleotide primer set according to the invention. The template nucleic acid can be selected from the group consisting of chromosomal DNA, cDNA, and a fragment thereof.

In the method, the target sequence can be labeled with a detectable labeling material. For example, the labeling material can be a fluorescent material, a phosphorescent material, or a radioactive material. Preferably, the labeling material can be the fluorophores, Cy-5 or Cy-3.

In the method, the probe set can be immobilized on a microarray substrate.

In the method, hybridization between the target sequence and the oligonucleotide probe can be performed under a high stringency hybridization condition. For example, the high stringency hybridization condition can be a 0.12M phosphate buffer including equal moles of Na₂HPO₄ and NaH₂PO₄, 1 mM EDTA, and 0.02% sodium dodecylsulfate and 65° C.

In the method, “PCR” refers to a polymerase chain reaction, a method for amplifying a target nucleic acid using a primer pair specifically binding with the target nucleic acid and a DNA polymerase. PCR is well known in the art. PCR can be performed using a commercially available kit. PCR can be classified into single PCR, i.e. the amplification of a single target sequence in a single PCR reaction, and multiplex PCR, i.e. the simultaneous amplification of multiple different target sequences in a single PCR reaction. Multiplex PCR is performed using a plurality of primer pairs, each of which is specific for a particular target sequence.

In the method, detection of the degree of hybridization between the oligonucleotide probe sequence and the target sequence can include labeling a PCR product (target sequence) with a detectable signal-emitting material; hybridizing the labeled PCR product with the oligonucleotide probe set; and detecting a signal generated from the hybridization product. Any detectable signal-emitting labeling material known in the art can be used. For example, the detectable signal-emitting material can be a material with a detectable optical property or an electrical signal-emitting material, but the present invention is not limited thereto. The material with a detectable optical property may be a fluorescent material or a phosphorescent material. The fluorescent material may be fluorescein, Cy-5, or Cy-3. The PCR product can be labeled with the detectable signal-emitting material before or after hybridization with the probe.

Detection of the hybrids does not require that the PCR product be labeled. For the instance in which the PCR product is unlabeled, hybridization between the PCR product and the oligonucleotide probe set can be detected by a difference in an electrical signal before and after hybridization. An example of a suitable electrical signal is capacitance, but the present invention is not limited thereto.

Hereinafter, the present invention will be described more specifically with reference to the following working examples. The following working examples are for illustrative purposes and are not intended to limit the scope of the invention.

### EXAMPLES

### Example 1

### Selection of Primers for Amplifying Target Sequences Commonly Found in Nine Respiratory Disease-Associated Bacterial Species

In Example 1, target sequences common to the 23S rRNA genes of nine bacterial species, i.e., Chlamydophila pneumoniae, Haemophilus influenzae, Mycoplasma pneumoniae, Klebsiella pneumoniae, Legionella pneumophila, Moraxella catarrhalis, Pseudomonas aeruginosa, Staphylococcus aureus, and Streptococcus pneumoniae were selected, and primer sets capable of amplifying the target sequences were designed.

First, sequences of respiratory disease-associated bacteria were acquired by sequencing 23s rRNAs obtained from clinical isolates. The bacterial species, number of strains of each bacterial species, and the SEQ ID Nos. for the sequences are presented in Table 2 below.

TABLE 2

| | The number | Examples of SEQ ID |
|---|---|---|
| Bacterial species | of strains | Nos: |
| Chlamydophila pneumoniae | 1 | 36, 37 |
| Haemophilus influenzae | 50 | 38 to 45 |
| Mycoplasma pneumoniae | 1 | 46 |
| Klebsiella pneumoniae | 61 | 47 to 61 |
| Legionella pneumophila | 22 | 62 to 64 |
| Moraxella catarrhalis | 10 | 65 to 67 |
| Pseudomonas aeruginosa | 72 | 68 to 76 |
| Staphylococcus aureus | 79 | 77 to 83 |
| Streptococcus pneumoniae | 105 | 84 to 94 |
| Total | 401 | — |

Conserved regions of the 23S rRNA genes of the bacterial species were selected using the program DNASTAR.

An oligonucleotide primer set was designed for each of the four target sequences using DNASTAR: an oligonucleotide primer set consisting of SEQ ID NOS: 1 and 2, an oligonucleotide primer set consisting of SEQ ID NOS: 3 and 4, an oligonucleotide primer set consisting of SEQ ID NOS: 5 and 6, and an oligonucleotide primer set consisting of SEQ ID NOS: 8 and 9. Each of these oligonucleotide primer sets can amplify a target region in the 23S rRNA genes of one or more of the nine bacterial species.

### Example 2

### Amplification of 23S rRNA Genes of Nine Respiratory Disease-Associated Bacterial Species Using Primer Sets of the Present Invention

The 23S rRNA genes of the nine respiratory disease-associated bacterial species were amplified using the four primer sets designed in Example 1.

First, amplification using polymerase chain reaction (“PCR”) was performed using only a single set of the primers designed in Example 1 (“single PCR”) to determine if each target sequence was specifically amplified. In the various single PCR, genomic DNA from 47 control bacterial species and 9 test bacterial species was used as template DNA. The control and test bacterial species used are listed in Table 4 below. Each single PCR was performed using 20 μl of a PCR solution containing 2 μl of a genomic DNA (extracted using a G-SPIN genomic DNA extraction kit; iNtRON), 1.5 mM MgCl₂, 250 mM of each dNTP, 10 mM tris-HCl (pH 9.0), 1 unit of Taq polymerase, and about 2 pmol of each primer, for 29 minutes and 5 seconds, as follows: 25 cycles of denaturation at 95° C. for 10 seconds, annealing at 60° C. for 10 seconds, and extension at 60° C. for 13 seconds.

The PCR products were identified by electrophoresis. The results for the 9 test species are presented in Table 3 below. Expected PCR products are amplified in a control PCR using gDNA from one of 47 control species (data not shown).

Next, multiplex PCR was performed using the four primer sets designed in Example 1 simultaneously to amplify a genomic DNA from one of the nine respiratory disease-associated bacterial species. The products from the multiplex PCR were identified by electrophoresis on an agarose gel.

(1) Preparation of Bacterial Cultures

Cultured isolates of the nine respiratory disease-associated bacterial species from the Asian-Pacific Research Foundation for Infectious Diseases (ARFID) were used.

(2) Multiplex PCR

The PCR mix for the multiplex PCR was made up to a total volume of 50 μl, containing 10.5 μl distilled water, 7.5 μl 10× buffer (750 mM Tris-HCl (pH9), 150 mM Ammonium Sulfate (NH₄)₂SO₄, 25 mM MgCl₂, 1 mg/ml BSA), 1 μl 200 μM dNTP (each), 20 μl 400 nM end-labeled primer (each, Bioneer, Korea), 5 μl extracted genomic DNA, and 1 μl Taq polymerase (5 units). Human MODY exon 9 (“e9”) DNA was used as a positive control.

The multiplex PCR was performed as follows: initial denaturation at 95° C. for one minute; 25 cycles of denaturation at 95° C. for 5 seconds, annealing at 62° C. for 13 seconds, and extension at 72° C. for 15 seconds; and extension at 72° C. for one minute.

The single PCR results and the multiplex PCR results for the 9 test bacterial species are presented in Table 3 below. Abbreviations used for the nine bacterial species in Table 3 are defined in Table 4 below. In Table 3, “PTC” is a positive control, and a primer set targeting 16S rRNA (16S-F and 16S-R) was also included as a control.

TABLE 3

| | Primer ID | Cpn | Hin | Kpn | Lpn | Mca | Mpn | Pae | Sau | Spn |
|---|---|---|---|---|---|---|---|---|---|---|
| The number | S01-F | 8 | 8 | 8 | 9 | 8 | 8 | 9 | 0 | 8 |
| of | S01-R | 9 | 8 | 10 | 10 | 10 | 8 | 11 | 0 | 10 |
| mismatched | A02-F | 5 | 0 | 0 | 0 | 0 | 7 | 0 | 5 | 5 |
| bases | A02-R | 3 | 0 | 0 | 0 | 1 | 6 | 0 | 1 | 1 |
| between | A07-F | 0 | 0 | 0 | 0 | 0 | 3 | 0 | 0 | 0 |
| primer | A07-R | 5 | 0 | 0 | 0 | 0 | 2 | 0 | 0 | 0 |
| and | A17-F | 0 | 0 | 0 | 0 | 0 | 0 | 0 | 0 | 1 |
| target | A17-R | 0 | 0 | 0 | 0 | 0 | 0 | 0 | 2 | 0 |
| sequence | 16S-F | 0 | 0 | 0 | 0 | 0 | 0 | 0 | 0 | 0 |
| | 16S-R | 0 | 0 | 0 | 0 | 0 | 0 | 0 | 0 | 0 |
| Single PCR | S01 | X | X | X | X | X | X | X | ◯ | X |
| results | A02 | ◯ | ◯ | ◯ | ◯ | ◯ | X | ◯ | X | X |
| | A07 | ◯ | ◯ | ◯ | ◯ | ◯ | ◯ | ◯ | ◯ | ◯ |
| | A17 | ◯ | ◯ | ◯ | ◯ | ◯ | ◯ | ◯ | ◯ | ◯ |
| | 16S | ◯ | ◯ | ◯ | ◯ | ◯ | ◯ | ◯ | ◯ | ◯ |
| Multiplex | S01 | X | X | X | X | X | X | X | ◯ | X |
| PCR results | A02 | X | ◯ | ◯ | ◯ | ◯ | X | ◯ | X | X |
| | A07 | X | ◯ | ◯ | ◯ | ◯ | X | ◯ | ◯ | ◯ |
| | A17 | ◯ | ◯ | ◯ | ◯ | ◯ | ◯ | ◯ | X | ◯ |
| | 16S | ◯ | ◯ | ◯ | ◯ | ◯ | ◯ | ◯ | ◯ | ◯ |
| | PTC | ◯ | ◯ | ◯ | ◯ | ◯ | ◯ | ◯ | ◯ | ◯ |

◯: band detection; X: no band detection

The single PCR results in Table 3 show that not all four target sequences were present in each of the test bacterial species. Further, the multiplex PCR results in Table 3 show that the target sequence of each of the four primer sets could be identified by multiplex PCR using the four primer sets, although not all four target sequences were present in each of the test bacterial species. This shows that, in general, the target sequence specificity of any one of the four primer sets was not affected by the presence of the other primer sets during the multiplex PCR. However, amplification of Chlamydophila pneumoniae using the primer sets A02-F and R and A07-F and R, Staphylococcus aureus using the primer set A17-F and R, and Streptococcus pneumoniae using the primer set A02-F and R, produced weak gel bands in the single PCR, that were not seen in the multiplex PCR.

FIG. 2 shows the result of electrophoretic analysis of PCR products obtained by multiplex PCR using the four primer sets of the present invention simultaneously with each of the nine test bacterial species.

### Example 3

### Detection of Nine Respiratory Disease-Associated Bacterial Species Using Microarrays

In Example 3, multiplex PCR products obtained as in Example 2 were allowed to hybridize with probes immobilized on microarrays and the degree of probe-target hybridization was determined to detect the presence of any PCR product amplified from a specific bacterial species.

Sample preparation and multiplex PCR were performed in the same manner as in Example 2 except that 5′-ends of all forward and reverse primers were labeled with Cy-3. PCR products were detected using microarrays as follows.

401 strains of the nine bacterial species were used as a test group and 226 strains of 47 species of 10 other bacterial genera were used as a control group.

The bacterial strains used as the test group and the control group are summarized in Table 4 below.

TABLE 4

| | Genus | Species | Abbreviation | The number of strains |
|---|---|---|---|---|
| Test group | Streptococcus | Streptococcus pneumoniae | Spn | 105 |
| | Staphylococcus | Staphylococcus aureus | Sau | 79 |
| | Klebsiella | Klebsiella pneumoniae | Kpn | 61 |
| | Pseudomonas | Pseudomonas aeruginosa | Pae | 72 |
| | Haemophilus | Haemophilus influenza | Hin | 50 |
| | Legionella | Legionella pneumophila | Lpn | 22 |
| | Moraxella | Moraxella catarrhalis | Mca | 10 |
| | Mycoplasma | Mycoplasma pneumoniae | Mpn | 1 |
| | Chlamydophila | Chlamydophila pneumoniae | Cpn | 1 |
| Total | 9 | 9 | | 401 |
| Control group | Acinetobacter | Acinetobacter baumannii | Aba | 9 |
| | | Acinetobacter calcoaceticus | Aca | 1 |
| | | Acinetobacter lwoffii | Alw | 1 |
| | Bacillus | Bacillus subtilis | Bsu | 1 |
| | Bordetella | Bordetella ansorpii | Ban | 1 |
| | | Bordetella avium | Bav | 1 |
| | | Bordetella bronchiseptica | Bbr | 1 |
| | Citrobacter | Citrobacter freundii | Cfr | 6 |
| | Enterobacter | Enterobacter aerogenes | Eae | 9 |
| | | Enterobacter cloacae | Ecl | 6 |
| | Enterococcus | Enterococcus faecalis | Efs | 11 |
| | | Enterococcus faecium | Efm | 11 |
| | Escherichia | Escherichia coli | Eco | 20 |
| | Gemella | Gemella melitensis | Gme | 1 |
| | Haemophilus | Haemophilus aprophilus | Hap | 1 |
| | Klebsiella | Klebsiella oxytoca | Kox | 12 |
| | Moraxella | Moraxella nonliquefaciens | Mon | |
| | Morganella | Morganella morganii | Mmo | 7 |
| | Proteus | Proteus mirabilis | Pmi | 8 |
| | | Proteus vulgaris | Pvu | 5 |
| | Pseudomonas | Pseudomonas fluorescens | Pfl | 9 |
| | | Pseudomonas putida | Ppu | 1 |
| | | Pseudomonas stutzeri | Pst | 1 |
| | Salmonella | Salmonella typhi | Sty | 10 |
| | | Salmonella murium | Stym | 7 |
| | Shigella | Shigella boydii | Sbo | 2 |
| | | Shigella flexneri | Sfl | 9 |
| | | Shigella sonnei | Sso | 6 |
| | Staphylococcus | Staphylococcus conii | Sco | 2 |
| | | Staphylococcus epidermis | Sep | 7 |
| | | Staphylococcus galinarium | Sga | 1 |
| | | Staphylococcus haemolyticus | Sha | 1 |
| | | Staphylococcus hominis | Sho | 3 |
| | | Staphylococcus intermedius | Sin | 2 |
| | | Staphylococcus lentus | Sle | 3 |
| | | Staphylococcus xylosus | Sxy | 1 |
| | | Coagulase negative staphylococci | CNS | 20 |
| | Streptococcus | Streptococcus agalactiae (Group B) | Sag | 1 |
| | | Streptococcus disgalactiae | Sdy | 1 |
| | | Streptococcus gordonii | Sgo | 1 |
| | | Streptococcus intermedius/milleri | St. in | 1 |
| | | Streptococcus mitis | Smi | 6 |
| | | Streptococcus oralis | Sor | 11 |
| | | Streptococcus pyogenes | Spy | 1 |
| | | Streptococcus salivarius subsp. thermophilus | Ssa | 1 |
| | | Streptococcus sanguinis | Ssan | 1 |
| | | Streptococcus suis | Ssu | 1 |
| Total | 18 | 47 | | 223 |

(1) Probe Design

Probes were selected from the PCR-amplified regions of the bacterial genomes using the program, DNASTAR. Probe information is summarized in Table 5.

TABLE 5

| Probe name | Target bacteria species | SEQ ID NO | Target region of 23S rRNA |
|---|---|---|---|
| A17-Chl.pneumo-re | Chlamydophila pneumoniae | 10 | 3041-3198 (A017) |
| A02-Hae.influe | Haemophilus influenzae | 11 | 853-1353 (A02) |
| A02-Hae.influe1-re | Haemophilus influenzae | 12 | 853-1353 (A02) |
| A02-H.inf2-I | Haemophilus influenzae | 13 | 853-1353 (A02) |
| A02-H.inf2-A | Haemophilus influenzae | 14 | 853-1353 (A02) |
| A07-Hae.influe | Haemophilus influenzae | 15 | 2483-2932 (A07) |
| A07-Kle.pneumo1-re | Klebsiella pneumoniae | 16 | 2483-2932 (A07) |
| A07-Kle.pneumo3-re | Klebsiella pneumoniae | 17 | 2483-2932 (A07) |
| A07-Kle.pneumo-re | Klebsiella pneumoniae | 18 | 2483-2932 (A07) |
| A02-Leg.pneumo | Legionella pneumophila | 19 | 853-1353 (A02) |
| A07-Leg.pneumo2 | Legionella pneumophila | 20 | 2483-2932 (A07) |
| A02-Mor.catarr1-re | Moraxella catarrhalis | 21 | 853-1353 (A02) |
| A02-Mor.cat2-C | Moraxella catarrhalis | 22 | 853-1353 (A02) |
| A02-Mor.cat2-N | Moraxella catarrhalis | 23 | 853-1353 (A02) |
| A07-Mor.catarr-re | Moraxella catarrhalis | 24 | 2483-2932 (A07) |
| A07-Mor.cat2-C | Moraxella catarrhalis | 25 | 2483-2932 (A07) |
| A07-Mor.cat2-N | Moraxella catarrhalis | 26 | 2483-2932 (A07) |
| A17-Myc.pneumo | Mycoplasma pneumoniae | 27 | 3041-3198 (A17) |
| A02-Pse.aerugi | Pseudomonas aeruginosa | 28 | 853-1353 (A02) |
| A07-Pse.aerugi-re | Pseudomonas aeruginosa | 29 | 2483-2932 (A07) |
| S01_Sta.aureu1 | Staphylococcus aureus | 30 | 124-365 (S01) |
| S01_Sta.aureu2 | Staphylococcus aureus | 31 | 124-365 (S01) |
| A07-Str.pneumo2-re | Streptococcus pneumoniae | 32 | 2483-2932(A07) |
| A07-Str.pneumo3 | Streptococcus pneumoniae | 33 | 2483-2932(A07) |
| A07-Str.pneumo3-O-re | Streptococcus pneumoniae | 34 | 2483-2932(A07) |
| A07-Str.pneumo3-P-re | Streptococcus pneumoniae | 35 | 2483-2932(A07) |

(2) Manufacturing of Probe-Immobilized Microarrays

Wafers were spin-coated with a solution of GAPTES (γ-aminopropyltriethoxy silane) (20%(v/v)) or GAPDES (γ-aminopropyldiethoxysilane) (20% (v/v)) in ethanol. The spin coating was performed using a spin coater (Model CEE 70, CEE) as follows: initial coating at a rate of 500 rpm/10 sec and main coating at a rate of 2000 rpm/10 sec. After the spin coating was completed, the wafers were placed in a Teflon wafer carrier and cured at 120° C. for 40 minutes. The cured wafers were immersed in water for 10 minutes, ultrasonically washed for 15 minutes, immersed in water for 10 minutes, and dried. The drying was performed using a spin-drier. All the experiments were conducted in a clean room class 1000 where most dust particles had been sufficiently removed.

A probe set including probes consisting of sequences as set forth in SEQ ID NOS: 10-35 was immobilized on the amino-activated wafers using a spotting method to thereby obtain microarrays.

(3) Detection

The PCR products were added directly to the microarrays. The microarrays were incubated at 42° C. for one hour so that probe-target hybridization occurred. The microarray was then washed with a washing buffer. Fluorescence intensity from hybridized PCR products was measured using a GenePix Scanner (Molecular Device, U.S.A.). The measured fluorescence intensity from a hybrid formed with a particular probe on the microarray is summarized in Table 6 below.

TABLE 6

| Numbers | Probe ID | Cpn | Hin | Kpn | Lpn | Mca | Mpn | Pae | Sau | Spn |
|---|---|---|---|---|---|---|---|---|---|---|
| 1 | per_probe4 | 65439 | 65351 | 65431 | 65334 | 65445 | 65445 | 65438 | 54165 | 62947 |
| 2 | spike_probe1 | 5968 | 5331 | 10141 | 4961 | 5562 | 5202 | 6666 | 3798 | 4446 |
| 3 | (−) | 109 | 84 | 89 | 85 | 99 | 106 | 102 | 90 | 96 |
| 4 | A02-IC1 | 2551 | 65281 | 23194 | 65327 | 61414 | 2376 | 65434 | 10813 | 1595 |
| 5 | A07-1C2 | 8275 | 51148 | 41926 | 56620 | 23938 | 10793 | 43631 | 3600 | 53214 |
| 6 | A07-IC3 | 21576 | 63581 | 65397 | 49212 | 31223 | 17463 | 62600 | 3316 | 5485 |
| 7 | A17-IC1 | 4361 | 3048 | 8117 | 1025 | 651 | 3339 | 3996 | 1232 | 52850 |
| 8 | A17-IC2 | 17227 | 27504 | 18664 | 35614 | 19881 | 6017 | 29337 | 1602 | 9037 |
| 9 | A17-IC3 | 42934 | 311 | 811 | 352 | 216 | 381 | 311 | 259 | 228 |
| 10 | CP_F2-4 | 65442 | 188 | 201 | 199 | 132 | 410 | 186 | 149 | 256 |
| 11 | A17-Chl.pneumo-re | 24543 | 583 | 766 | 588 | 1911 | 764 | 580 | 352 | 1621 |
| 12 | HI_F2-4 | 544 | 65417 | 278 | 504 | 205 | 572 | 1143 | 214 | 484 |
| 13 | A02-Hae.influe | 3036 | 65350 | 482 | 658 | 376 | 2650 | 694 | 818 | 1967 |
| 14 | A07-Hae.influe | 199 | 65353 | 1064 | 941 | 24009 | 172 | 3913 | 845 | 632 |
| 15 | A02-Hae.influe1-r | 1923 | 65339 | 3128 | 586 | 5365 | 1733 | 751 | 639 | 1224 |
| 16 | KP_F2-4 | 5528 | 1169 | 65343 | 1726 | 989 | 5368 | 2821 | 1732 | 3445 |
| 17 | A07-Kle.pneumo1-r | 143 | 1017 | 34399 | 5176 | 151 | 208 | 1072 | 4581 | 11154 |
| 18 | A07-Kle.pneumo3-r | 132 | 163 | 48651 | 137 | 133 | 146 | 157 | 139 | 158 |
| 19 | A07-Kle.pneumo-re | 155 | 273 | 52375 | 139 | 659 | 154 | 156 | 148 | 143 |
| 20 | LP_F1-1 | 236 | 397 | 335 | 65339 | 216 | 211 | 276 | 192 | 186 |
| 21 | A02-Leg.pneumo | 386 | 10205 | 560 | 65290 | 28278 | 242 | 2853 | 159 | 331 |
| 22 | A07-Leg.pneumo2 | 134 | 129 | 106 | 51306 | 123 | 140 | 140 | 111 | 136 |
| 23 | MC_F2-2 | 295 | 199 | 221 | 189 | 65433 | 250 | 211 | 192 | 264 |
| 24 | A02-Mor.catarr1-r | 283 | 190 | 192 | 159 | 65435 | 209 | 154 | 139 | 159 |
| 25 | A07-Mor.catarr-re | 156 | 177 | 154 | 776 | 65435 | 168 | 158 | 137 | 176 |
| 26 | MP_F1-4 | 214 | 306 | 254 | 679 | 1252 | 65438 | 4059 | 252 | 258 |
| 27 | A17-Myc.pneumo | 573 | 858 | 767 | 550 | 2483 | 65441 | 489 | 299 | 365 |
| 28 | PA_F1-1 | 719 | 598 | 764 | 984 | 1510 | 546 | 65435 | 450 | 416 |
| 29 | A02-Pse.aerugi | 144 | 138 | 109 | 126 | 121 | 151 | 46381 | 124 | 157 |
| 30 | A07-Pse.aerugi-re | 181 | 158 | 275 | 40194 | 5333 | 176 | 28993 | 147 | 171 |
| 31 | S01-IC1 | 596 | 404 | 659 | 751 | 325 | 472 | 478 | 57783 | 519 |
| 32 | SA_F1-2 | 549 | 2256 | 906 | 371 | 496 | 529 | 593 | 65439 | 3883 |
| 33 | S01_Sta.aureu1 | 953 | 595 | 797 | 515 | 404 | 829 | 728 | 65435 | 676 |
| 34 | S01_Sta.aureu2 | 943 | 591 | 845 | 499 | 447 | 877 | 702 | 65416 | 638 |
| 35 | SP_F2-3 | 1921 | 1146 | 1443 | 773 | 626 | 1495 | 1306 | 428 | 65427 |
| 36 | A07-Str.pneumo | 10864 | 11006 | 14848 | 9588 | 6890 | 9306 | 9166 | 5435 | 65426 |
| 37 | A07-Str.pneumo2-r | 238 | 121 | 155 | 99 | 117 | 171 | 154 | 3056 | 65427 |
| 38 | A07-Str.pneumo3 | 282 | 187 | 265 | 160 | 161 | 198 | 204 | 200 | 64541 |
| 39 | A07-Str.pneumo3-O | 183 | 138 | 150 | 84 | 122 | 154 | 140 | 130 | 50128 |
| 40 | A07-Str.pneumo3-P | 201 | 117 | 132 | 338 | 118 | 169 | 136 | 116 | 63492 |

As shown in Table 6, the nine bacterial species were detected with high sensitivity using the probes designed according to the present invention. Probes corresponding to numbers 1-10, 31 and given in Table 6 represent various control probes. A probe corresponding number 1 is a positive PCR probe indicating that a PCR is successfully conducted. A probe corresponding number 2 is a positive microarray probe indicating that a hybridization in successfully conducted. A probe corresponding number 3 is a negative probe. A probes corresponding numbers 4-10 are a positive probe indicating that each target region corresponding to each region is successfully amplified.

The results of Table 6 also reveal that the probes designed according to the present invention had 99.8 or higher % specificity and 100% sensitivity for the nine target bacterial species among 640 strains of 56 bacterial species as shown in Table 7 to 16. The experimental data obtained by conducting a hybridization on a microarray were confirmed by culturing experiment.

TABLE 7

Culture

| | | |
|---|---|---|
| S. pneumoniae | + | − |

| | | | |
|---|---|---|---|
| Microarray | + | 104 | 0 |
| | − | 0 | 518 |
| Sensitivity | Specificity | 100% | 100% |

TABLE 8

Culture

| | | |
|---|---|---|
| H. influenzae | + | − |

| | | | |
|---|---|---|---|
| Microarray | + | 48 | 0 |
| | − | 0 | 574 |
| Sensitivity | Specificity | 100% | 100% |

TABLE 9

Culture

| | | |
|---|---|---|
| S. aureus | + | − |

| | | | |
|---|---|---|---|
| Microarray | + | 78 | 0 |
| | − | 0 | 544 |
| Sensitivity | Specificity | 100% | 100% |

TABLE 10

Culture

| | | |
|---|---|---|
| P. aeruginosa | + | − |

| | | | |
|---|---|---|---|
| Microarray | + | 72 | 0 |
| | − | 0 | 550 |
| Sensitivity | Specificity | 100% | 100% |

TABLE 11

Culture

| | | |
|---|---|---|
| K. pneumoniae | + | − |

| | | | |
|---|---|---|---|
| Microarray | + | 61 | 1 |
| | − | 0 | 561 |
| Sensitivity | Specificity | 100% | 99.8% |

TABLE 12

Culture

| | | |
|---|---|---|
| M. catarrhalis | + | − |

| | | | |
|---|---|---|---|
| Microarray | + | 10 | 1 |
| | − | 0 | 612 |
| Sensitivity | Specificity | 100% | 99.8% |

TABLE 13

Culture

| | | |
|---|---|---|
| C. pneumoniae | + | − |

| | | | |
|---|---|---|---|
| Microarray | + | 1 | 0 |
| | − | 0 | 621 |
| Sensitivity | Specificity | 100% | 100% |

TABLE 14

Culture

| | | |
|---|---|---|
| L. pneumophila | + | − |

| | | | |
|---|---|---|---|
| Microarray | + | 23 | 0 |
| | − | 0 | 599 |
| Sensitivity | Specificity | 100% | 100% |

TABLE 15

Culture

| | | |
|---|---|---|
| Total (9 bacterial species) | + | − |

| | | | |
|---|---|---|---|
| Microarray | + | 402 | 2 |
| | − | 0 | 218 |
| Sensitivity | Specificity | 100% | 99.1% |

TABLE 16

Culture

| | | |
|---|---|---|
| M. pneumoniae | + | − |

| | | | |
|---|---|---|---|
| Microarray | + | 5 | 0 |
| | − | 0 | 617 |
| Sensitivity | Specificity | 100% | 100% |

A nucleic acid primer set of the invention can amplify target sequence(s) derived from nine respiratory disease-associated bacterial species.

A probe set of the present invention is specific to a target sequence of a PCR product amplified using the primer set of the invention, and thus, can be used for detection of at least one of the nine respiratory disease-associated bacterial species.

A microarray of the invention can be used for detection of at least one of the nine respiratory disease-associated bacterial species.

A detection method of the invention ensures high-efficiency and high-specificity of detection of the nine respiratory disease-associated bacterial species.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An oligonucleotide primer set comprising at least one oligonucleotide set selected from the group consisting of:
an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2;
an oligonucleotide set comprising an oligonucleotide consisting of at least 10 contiguous nucleotides of SEQ ID NO: 3 and an oligonucleotide consisting of at least 10 contiguous nucleotides of SEQ ID NO: 4;
an oligonucleotide set comprising an oligonucleotide consisting of at least 10 contiguous nucleotides of SEQ ID NO: 5 and an oligonucleotide consisting of at least 10 contiguous nucleotides of a sequence selected from the group consisting of SEQ ID NOS: 6 and 7; and
an oligonucleotide set comprising an oligonucleotide consisting of at least 10 contiguous nucleotides of SEQ ID NO: 8 and an oligonucleotide consisting of at least 10 contiguous nucleotides of SEQ ID NO: 9.

2. The oligonucleotide primer set of claim 1, comprising:
the oligonucleotide set comprising the oligonucleotide consisting of at least 10 contiguous nucleotides of SEQ ID NO: 1 and the oligonucleotide consisting of at least 10 contiguous nucleotides of SEQ ID NO: 2;
the oligonucleotide set comprising the oligonucleotide consisting of at least 10 contiguous nucleotides of SEQ ID NO: 3 and the oligonucleotide consisting of at least 10 contiguous nucleotides of SEQ ID NO: 4;
the oligonucleotide set comprising the oligonucleotide consisting of at least 10 contiguous nucleotides of SEQ ID NO: 5 and the oligonucleotide consisting of at least 10 contiguous nucleotides of a sequence selected from the group consisting of SEQ ID NOS: 6 and 7; and
the oligonucleotide set comprising the oligonucleotide consisting of at least 10 contiguous nucleotides of SEQ ID NO: 8 and the oligonucleotide consisting of at least 10 contiguous nucleotides of SEQ ID NO: 9.

3. The oligonucleotide primer set of claim 1, which comprises
the oligonucleotide set comprising an oligonucleotide consisting of at least 10 contiguous nucleotides of SEQ ID NO: 1 and an oligonucleotide consisting of at least 10 contiguous nucleotides of SEQ ID NO: 2.

4. The oligonucleotide primer set of claim 1, which comprises
the oligonucleotide set comprising an oligonucleotide consisting of at least 10 contiguous nucleotides of SEQ ID NO: 3 and an oligonucleotide consisting of at least 10 contiguous nucleotides of SEQ ID NO: 4.

5. The oligonucleotide primer set of claim 1, which comprises
the oligonucleotide set comprising an oligonucleotide consisting of at least 10 contiguous nucleotides of SEQ ID NO: 5 and an oligonucleotide consisting of at least 10 contiguous nucleotides of a sequence selected from the group consisting of SEQ ID NOS: 6 and 7.

6. The oligonucleotide primer set of claim 1, which comprises
the oligonucleotide set comprising an oligonucleotide consisting of at least 10 contiguous nucleotides of SEQ ID NO: 8 and an oligonucleotide consisting of at least 10 contiguous nucleotides of SEQ ID NO: 9.

7. The oligonucleotide primer set of claim 1, which comprises
an oligonucleotide set comprising an oligonucleotide consisting of SEQ ID NO: 1 and an oligonucleotide consisting of SEQ ID NO: 2;
an oligonucleotide set comprising an oligonucleotide consisting of SEQ ID NO: 3 and an oligonucleotide consisting of SEQ ID NO: 4;
an oligonucleotide set comprising an oligonucleotide consisting of SEQ ID NO: 5 and an oligonucleotide consisting of SEQ ID NO: 6 or SEQ ID NO:7; and
an oligonucleotide set comprising an oligonucleotide consisting of SEQ ID NO: 8 and an oligonucleotide consisting of SEQ ID NO: 9.

8. An oligonucleotide probe set comprising an oligonucleotide probe selected from the group consisting of:
an oligonucleotide probe comprising an oligonucleotide consisting of at least 10 contiguous nucleotides of SEQ ID NO: 10 or a complement of the oligonucleotide;
an oligonucleotide probe comprising an oligonucleotide consisting of at least 10 contiguous nucleotides of a sequence selected from the group consisting of SEQ ID NOS: 11-14 or a complement of the oligonucleotide;
an oligonucleotide probe comprising an oligonucleotide consisting of at least 10 contiguous nucleotides of SEQ ID NO: 15 or a complement of the oligonucleotide;
an oligonucleotide probe comprising an oligonucleotide consisting of at least 10 contiguous nucleotides of a sequence selected from the group consisting of SEQ ID NOS: 16-18 or a complement of the oligonucleotide;
an oligonucleotide probe comprising an oligonucleotide consisting of at least 10 contiguous nucleotides of SEQ ID NO: 19 or a complement of the oligonucleotide;
an oligonucleotide probe comprising an oligonucleotide consisting of at least 10 contiguous nucleotides of SEQ ID NO: 20 or a complement of the oligonucleotide;
an oligonucleotide probe an oligonucleotide consisting of at least 10 contiguous nucleotides of a sequence selected from the group consisting of SEQ ID NOS: 21-23 or a complement of the oligonucleotide;
an oligonucleotide probe comprising consisting of an oligonucleotide consisting of at least 10 contiguous nucleotides of a sequence selected from the group consisting of SEQ ID NOS: 24-26 or a complement of the oligonucleotide;
an oligonucleotide probe comprising an oligonucleotide consisting of at least 10 contiguous nucleotides of SEQ ID NO: 27 or a complement of the oligonucleotide;
an oligonucleotide probe comprising an oligonucleotide consisting of at least 10 contiguous nucleotides of SEQ ID NO: 28 or a complement of the oligonucleotide;
an oligonucleotide probe comprising an oligonucleotide consisting of at least 10 contiguous nucleotides of SEQ ID NO: 29 or a complement of the oligonucleotide;
an oligonucleotide probe comprising an oligonucleotide consisting of at least 10 contiguous nucleotides of a sequence selected from the group consisting of SEQ ID NOS: 30 and 31 or a complement of the oligonucleotide; and
an oligonucleotide probe comprising an oligonucleotide consisting of at least 10 contiguous nucleotides of a sequence selected from the group consisting of SEQ ID NOS: 32-35 or a complement of the oligonucleotide.

9. The oligonucleotide probe set of claim 8, comprising
the oligonucleotide consisting of at least 10 contiguous nucleotides of SEQ ID NO: 10 or a complement of the oligonucleotide.

10. The oligonucleotide probe set of claim 8, comprising an oligonucleotide probe selected from the group consisting of:
the oligonucleotide probe comprising the oligonucleotide consisting of at least 10 contiguous nucleotides of a sequence selected from the group consisting of SEQ ID NOS: 11-14 or the complement of the oligonucleotide; and
the oligonucleotide probe comprising the oligonucleotide consisting of at least 10 contiguous nucleotides of SEQ ID NO: 15 or the complement of the oligonucleotide.

11. The oligonucleotide probe set of claim 8, comprising
the oligonucleotide consisting of at least 10 contiguous nucleotides of a sequence selected from the group consisting of SEQ ID NOS: 16-18 or the complement of the oligonucleotide.

12. The oligonucleotide probe set of claim 8, comprising an oligonucleotide probe selected from the group consisting of:
the oligonucleotide probe comprising the oligonucleotide consisting of at least 10 contiguous nucleotides of SEQ ID NO: 19 or the complement of the oligonucleotide; and
the oligonucleotide probe comprising the oligonucleotide consisting of at least 10 contiguous nucleotides of SEQ ID NO: 20 or the complement of the oligonucleotide.

13. The oligonucleotide probe set of claim 8, comprising an oligonucleotide probe selected from the group consisting of:
the oligonucleotide probe comprising the oligonucleotide consisting of at least 10 contiguous nucleotides of a sequence selected from the group consisting of SEQ ID NOS: 21-23 or the complement of the oligonucleotide; and
the oligonucleotide probe comprising the oligonucleotide consisting of at least 10 contiguous nucleotides of a sequence selected from the group consisting of SEQ ID NOS: 24-26 or the complement of the oligonucleotide.

14. The oligonucleotide probe set of claim 8, comprising
the oligonucleotide consisting of at least 10 contiguous nucleotides of SEQ ID NO: 27 or the complement of the oligonucleotide.

15. The oligonucleotide probe set of claim 8, comprising an oligonucleotide probe selected from the group consisting of:
the oligonucleotide probe comprising the oligonucleotide consisting of at least 10 contiguous nucleotides of SEQ ID NO: 28 or the complement of the oligonucleotide; and
the oligonucleotide probe comprising the oligonucleotide consisting of at least 10 contiguous nucleotides of SEQ ID NO: 29 or the complement of the oligonucleotide.

16. The oligonucleotide probe set of claim 8, comprising
the oligonucleotide consisting of at least 10 contiguous nucleotides of a sequence selected from the group consisting of SEQ ID NOS: 30 and 31 or the complement of the oligonucleotide.

17. The oligonucleotide probe set of claim 8, comprising
the oligonucleotide consisting of at least 10 contiguous nucleotides of a sequence selected from the group consisting of SEQ ID NOS: 32-35 or the complement of the oligonucleotide.

18. The oligonucleotide probe set of claim 8, comprising an oligonucleotide probe selected from the group consisting of:
the oligonucleotide probe comprising the oligonucleotide consisting of SEQ ID NO: 10, or the complement thereof;
the oligonucleotide probe comprising the oligonucleotide consisting of a sequence selected from the group consisting of SEQ ID NOS: 11-14, or the complement thereof;
the oligonucleotide probe comprising the oligonucleotide consisting of SEQ ID NO: 15, or the complement thereof;
the oligonucleotide probe comprising the oligonucleotide consisting of a sequence selected from the group consisting of SEQ ID NOS: 16-18, or the complement thereof;
the oligonucleotide probe comprising the oligonucleotide consisting of SEQ ID NO: 19, or the complement thereof;
the oligonucleotide probe comprising the oligonucleotide consisting of SEQ ID NO: 20, or the complement thereof;
the oligonucleotide probe comprising the oligonucleotide consisting of a sequence selected from the group consisting of SEQ ID NOS: 21-23, or the complement thereof;
the oligonucleotide probe comprising the oligonucleotide consisting of a sequence selected from the group consisting of SEQ ID NOS: 24-26, or the complement thereof;
the oligonucleotide probe comprising the oligonucleotide consisting of SEQ ID NO: 27, or the complement thereof;
the oligonucleotide probe comprising the oligonucleotide consisting of SEQ ID NO: 28, or the complement thereof;
the oligonucleotide probe comprising the oligonucleotide consisting of SEQ ID NO: 29, or the complement thereof;
the oligonucleotide probe comprising the oligonucleotide consisting of a sequence selected from the group consisting of SEQ ID NOS: 30 and 31, or the complement thereof; and
the oligonucleotide probe comprising the oligonucleotide consisting of a sequence selected from the group consisting of SEQ ID NOS: 32-35, or the complement thereof.

19. A microarray, comprising
a substrate,
wherein the oligonucleotide probe set of claim 8 is immobilized on the substrate.

20. A method of detecting a bacterial species selected from the group consisting of Chlamydophila pneumoniae, Haemophilus influenza, Klebsiella pneumoniae, Legionella pneumophila, Moraxella catarrhalis, Mycoplasma pneumoniae, Pseudomonas aeruginosa, Staphylococcus aureus, and Streptococcus pneumoniae, the method comprising:
contacting a sample to the oligonucleotide probe set of claim 8 so that an oligonucleotide probe hybridizes with a target sequence present in the sample; and
detecting a degree of hybridization between the oligonucleotide probe and the target sequence.

21. The method of claim 20, wherein the sample comprises a PCR product.

22. The method of claim 21, wherein the PCR product is obtained by PCR using
template DNA obtained from a bacterial species selected from the group consisting of Chlamydophila pneumoniae, Haemophilus influenza, Klebsiella pneumoniae, Legionella pneumophila, Moraxella catarrhalis, Mycoplasma pneumoniae, Pseudomonas aeruginosa, Staphylococcus aureus, and Streptococcus pneumoniae, and
an oligonucleotide primer set comprising an oligonucleotide set selected from the group consisting of:
an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2;
an oligonucleotide set comprising an oligonucleotide consisting of at least 10 contiguous nucleotides of SEQ ID NO: 3 and an oligonucleotide consisting of at least 10 contiguous nucleotides of SEQ ID NO: 4;
an oligonucleotide set comprising an oligonucleotide consisting of at least 10 contiguous nucleotides of SEQ ID NO: 5 and an oligonucleotide consisting of at least 10 contiguous nucleotides of a sequence selected from the group consisting of SEQ ID NO: 6 and SEQ ID NO:7; and
an oligonucleotide set comprising an oligonucleotide consisting of at least 10 contiguous nucleotides of SEQ ID NO: 8 and an oligonucleotide consisting of oat least 10 contiguous nucleotides of SEQ ID NO: 9.

23. The method of claim 22, wherein the oligonucleotide primer set comprises
an oligonucleotide set comprising an oligonucleotide consisting of at least 10 contiguous nucleotides of SEQ ID NO: 1 and an oligonucleotide consisting of at least 10 contiguous nucleotides of SEQ ID NO: 2;
an oligonucleotide set comprising an oligonucleotide consisting of at least 10 contiguous nucleotides of SEQ ID NO: 3 and an oligonucleotide consisting of at least 10 contiguous nucleotides of SEQ ID NO: 4;
an oligonucleotide set comprising an oligonucleotide consisting of at least 10 contiguous nucleotides of SEQ ID NO: 5 and an oligonucleotide consisting of at least 10 contiguous nucleotides of a sequence selected from the group consisting of SEQ ID NO: 6 and SEQ ID NO:7; and
an oligonucleotide set comprising an oligonucleotide consisting of at least 10 contiguous nucleotides of SEQ ID NO: 8 and an oligonucleotide consisting of oat least 10 contiguous nucleotides of SEQ ID NO: 9.

24. The method of claim 20, wherein the target sequence is labeled with a detectable labeling material.

25. The method of claim 24, wherein the labeling material is a fluorescent material, a phosphorescent material, or a radioactive material.

26. The method of claim 20, wherein the oligonucleotide probe set is immobilized on a microarray substrate.


SEQUENCE LISTING (94 sequences)

SEQ ID NO: 1 - DNA, 20 bp, Artificial Sequence
  primer S01-F  forward primer for amplifying S01
gtcatggcttaccaaaccga

SEQ ID NO: 2 - DNA, 23 bp, Artificial Sequence
  primer S01-R reverse primer for amplifying SO1
aatgttgtctctcttgagtggat

SEQ ID NO: 3 - DNA, 23 bp, Artificial Sequence
  primer A02-F forward primer for amplifying A02
gcgtaccttttgtataatgggtc

SEQ ID NO: 4 - DNA, 19 bp, Artificial Sequence
  primer A02-R reverse primer for amplifying A02
cagaccgccagctaaggtc

SEQ ID NO: 5 - DNA, 19 bp, Artificial Sequence
  primer A07-F forward primer for amplifying A07
tgtcgggtaagttccgacc

SEQ ID NO: 6 - DNA, 19 bp, Artificial Sequence
  primer A07-R reverse primer for amplifying A07
tagtgatccggtggttcyg

SEQ ID NO: 7 - DNA, 19 bp, Artificial Sequence
  primer A07-R2 reverse primer for amplifying A07
tggaagggccatcgctcaa

SEQ ID NO: 8 - DNA, 20 bp, Artificial Sequence
  primer A17-F forward primer for amplifying A17
cacctcgatgtcgrctcatc

SEQ ID NO: 9 - DNA, 21 bp, Artificial Sequence
  primer A17-R reverse primer for amplifying A17
tgytyctagtacgagaggacc

SEQ ID NO: 10 - DNA, 25 bp, Artificial Sequence
  probe A17-Chl pneumo-re probe specific for A17
gacagtttggtctctatcctttgtg

SEQ ID NO: 11 - DNA, 23 bp, Artificial Sequence
  probe A02-Hae.influe specific for A02
accaaagagtgatactcaggaga

SEQ ID NO: 12 - DNA, 27 bp, Artificial Sequence
  probe A02-Hae.influe1-re specific for A02
aaactacgaataccaaagagtgatact

SEQ ID NO: 13 - DNA, 22 bp, Artificial Sequence
  probe A02-H.inf2-I specific for A02
caaactacgaataccaaagagt

SEQ ID NO: 14 - DNA, 22 bp, Artificial Sequence
  probe A02-H.inf2-A specific for A02
caaactgcgaataccgaagagt

SEQ ID NO: 15 - DNA, 23 bp, Artificial Sequence
  probe A07-Hae.influe specific for A07
taagcaagcttaactgcgagaca

SEQ ID NO: 16 - DNA, 23 bp, Artificial Sequence
  probe A07-Kle.pneumo1-re specific for A07
ataagctagcttgactgcgagcg

SEQ ID NO: 17 - DNA, 21 bp, Artificial Sequence
  probe A07-Kle.pneumo3-re specific for A07
gcgtggacgccagtctgcgtg

SEQ ID NO: 18 - DNA, 21 bp, Artificial Sequence
  probe A07-Kle.pneumo-re
gttggccccttaccggggttg

SEQ ID NO: 19 - DNA, 20 bp, Artificial Sequence
  probe A02 Leg.pneumo
gtcatggcttaccaaaccga

SEQ ID NO: 20 - DNA, 23 bp, Artificial Sequence
  probe A07-Leg.penumo2
ttggtccagtaatcctggatgag

SEQ ID NO: 21 - DNA, 24 bp, Artificial Sequence
  probe A02-Mor-catarr1-re specific for A02
ccgaataccgatgagtaatatccg

SEQ ID NO: 22 - DNA, 21 bp, Artificial Sequence
  probe A02-Mor.cat2-C specific for A02
tagggggtcacaccgacttac

SEQ ID NO: 23 - DNA, 21 bp, Artificial Sequence
  probe A02-Mor.cat2-N specific for A02
tagggggtcatcccgacttac

SEQ ID NO: 24 - DNA, 24 bp, Artificial Sequence
  probe A07-Mor.catarr-re specific for A07
tgttggggttctaacttaggatca

SEQ ID NO: 25 - DNA, 24 bp, Artificial Sequence
  probe A07-Mor-cat2-C specific for A07
ctaacttaggatcaacaaatccaa

SEQ ID NO: 26 - DNA, 24 bp, Artificial Sequence
  probe A07-Mor-cat2-N specific for A07
ctaacttaggattaacaaatccaa

SEQ ID NO: 27 - DNA, 23 bp, Artificial Sequence
  probe A17-Myc.pneumo specific for A17
acaggttggtccctatctattgt

SEQ ID NO: 28 - DNA, 20 bp, Artificial Sequence
  probe A02-Pse.aerugi specific for A02
ccactcccgttgaaaaggta

SEQ ID NO: 29 - DNA, 24 bp, Artificial Sequence
  probe A07-Pse.aerugi-re specific for A07
gcttgaggttctaactctggtccg

SEQ ID NO: 30 - DNA, 22 bp, Artificial Sequence
  probe S01-Sta.aureu1 specific for S01
acggagttacaaaggacgacat

SEQ ID NO: 31 - DNA, 22 bp, Artificial Sequence
  probe S01-Sta.aureu2 specific for S01
aaaggacgacattagacgaatc

SEQ ID NO: 32 - DNA, 20 bp, Artificial Sequence
  probe A07-Str.pneumon2-re
gacgctgttgggatactacc

SEQ ID NO: 33 - DNA, 20 bp, Artificial Sequence
  probe A07-Sta.pneumo3 specfic for A07
gccagtttcgaaggagacgc

SEQ ID NO: 34 - DNA, 20 bp, Artificial Sequence
  probe A07-Str.pneumo3-O-re specific for A07
tttcgaaggagacgttgttg

SEQ ID NO: 35 - DNA, 20 bp, Artificial Sequence
  probe A07-Str.pneumo3-P-re specific for A07
tttcgaaggagacgctgttg

SEQ ID NO: 36 - DNA, 1024 bp, Chlamydophila pneumoniae LKK-1
ttacctgcagtaatcttcggtgagctggtatagagctatgacccggaggtatccgaatggggcaacccgatagactaatagtctatcattatatgttgaatacataggcatataaggcgacacccgctgaactgaaacatcttagtaagcggaggaaaagaaatcaaagagattccctgtgtagcggcgagcgaaaggggaacagcctaaaccatatttttaatatggggttgtagggtcgataacatgggatcttaagttttagttgaatacttctggaaagttgaacgatacagggtgatagtcccgtaaacgaaaaaacaaaagacgctaatcgatacctgagtagggctagacacgtgaaacctagtctgaatctggggagaccactctccaaggctaaatactagtcaatgacctatagtgaaccagtactgtgaaggaaaggtgaaaagaacccttgttaagggagtgaaatagaacctgaaaccagtagcttataagcggtcggagacctataactcttcggagtaatggttgacggcgtgccttttgcatgatgagccagggagttaagttaaacggcgagattaagggatttacattccggagtcgaagcgaaagcgagttttaaaagagcgttttagtcgtttgatttagacacgaaaccaagtgagctatttatgaccaggttgaagcattggtaagactttgtggaggaccgaaccagtacatgttgaaaaatgtttggatgagttgtgaataggggtgaaaggccaatcaaacttggagatatcttgttctctccgaaataactttagggttagcctcggatattaagtttttgggggtagagcactgaattctagcgggggcctaccggcttaccaacggaaatcaaactccgaataccaaaagcgagtccgggagatagacagcgggggctaagcttcgttgtcgagaggggaacagcccagaccgccgattaaggtccctaattttatgctaagtgagtaaggaagtg

SEQ ID NO: 37 - DNA, 1024 bp, Chlamydophila pneumoniae TW-183
tacagaccaagttgttaagagctattggcggatgccttggcattgacaggcgatgaaggatgcgtttacctgcagtaatcttcggtgagctggtatagagctatgacccggaggtatccgaatggggcaacccgatagactaatagtctatcattatatgttgaatacataggcatataaggcgacacccgctgaactgaaacatcttagtaagcggaggaaaagaaatcaaagagattccctgtgtagcggcgagcgaaaggggaacagcctaaaccatatttttaatatggggttgtagggtcgataacatgggatcttaagttttagttgaatacttctggaaagttgaacgatacagggtgatagtcccgtaaacgaaaaaacaaaagacgctaatcgatacctgagtagggctagacacgtgaaacctagtctgaatctggggagaccactctccaaggctaaatactagtcaatgacctatagtgaaccagtactgtgaaggaaaggtgaaaagaacccttgttaagggagtgaaatagaacctgaaaccagtagcttataagcggtcggagacctataactcttcggagtaatggttgacggcgtgccttttgcatgatgagccagggagttaagttaaacggcgagattaagggatttacattccggagtcgaagcgaaagcgagttttaaaagagcgttttagtcgtttgatttagacacgaaaccaagtgagctatttatgaccaggttgaagcattggtaagactttgtggaggaccgaaccagtacatgttgaaaaatgtttggatgagttgtgaataggggtgaaaggccaatcaaacttggagatatcttgttctctccgaaataactttagggttagcctcggatattaagtttttgggggtagagcactgaattctagcgggggcctaccggcttaccaacggaaatcaaactccgaataccaaaagcgagtccgggagatagacagcgggggctaagcttcgttgtc

SEQ ID NO: 38 - DNA, 1024 bp, Haemophilus influenzae
  misc_feature (25): n is a, c, g, or t
ctgcgaaagcttggatgagtcgatnagaggcgtttaatccnagatgtccgaatggggaaacccagttgggtgaagaacccactatcattatctgaatccataggataatgaggcgaaccgggagaactgaaacatctaagtaccccgaggaaaagaaatcaaccgagatttcgttagtagcggcgagcgaacgcgaaggagcctgttagtgataatgacagagacagaggaacaagctgggaagcttggcgacacagggtgatagccccgtactcgaantccaggttatggtactaagctaacgacaagtaaggcgggacacgtgatatcctgtttgaagatggggggaccatcctccaaggctaaatactcctgattgaccgatagtgaaccagtactgtgaaggaaaggcgaaaagaaccccggtgaggggagtgaaatagaacctgaaaccttgtacgtacaagcagtgggagcctgaaagggtgactgcgtaccttttgtataatgggtcagcgacttatattttgtagcgaggttaaccgaataggggagccgaagggaaaccgagtcttaactgggcgagtagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaatctatttaggtagagccttgagtggacaccttcgggggtagagcactgtttcggctaggggtccatcccggattaccaacccgatgcaaactgcgaataccgaagagtgatactcaggagacacacggcgggtgctaacgttcgtcgtggagagggaaacaacccagaccgccagctaaggtcccaaagtctatattaagtgggaaacgaagtgggaaggcttagacagctaggatgttggcttagaagcagccatcatttaaagaaagc

SEQ ID NO: 39 - DNA, 1024 bp, Haemophilus influenzae
ctatctgcgaaaagcttggatgagtcgataagaggcgtttaatccaagatatccgaatggggaaacccagtagatgaagaatctactatcaacaagtgaattcatagcttgttgaggcaaaccgggagaactgaaacatctaagtaccccgaggaaaagaaatcaaccgagatttcgtcagtagtggcgagcgaaagcgaaagagccagtaagtgatagcagtatagttaggagaatgtgttgggaaacacaatcaaagagggtgataatcccgtatcttaaaaccatattgtggtactaagctaacgagaagtagggcgggacacgtgatatcctgtttgaagaaggggggaccatcctccaaggctaaatactcctgattgaccgatagtgaaccagtactgtgaaggaaaggcgaaaagaaccccggtgaggggagtgaaatagaacctgaaaccttgtacgtacaagcagtgggagccccatcacttaatccgattggaaagtgatgcggaaatagagcgcactttaaggtaaggcgaacgaagtgagacaaaccttcccaaaaaaaagcagtgggattaagtgatggggtgactgcgtaccttttgtataatgggtcagcgacttatattttgtagcgaggttaaccgaataggggagccgaagggaaaccgagtcttaactgggcgaagagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaatctatttaggtagagccttgaggtgacacctttgggggtagagcactgtttcggctaggggtccatcccggattaccaacccgatgcaaactacgaataccaaagagtgatactcaggagacacacggcgggtgctaacgtccgtcgtggagagggaaacaa

SEQ ID NO: 40 - DNA, 1024 bp, Haemophilus influenzae
ctatctgcgaaaagcttggatgagtcgataagaggcgtttaatccaagatatccgaatggggaaacccagtagatgaagaatctactatcaacaagtgaattcatagcttgttgaggcaaaccgggagaactgaaacatctaagtaccccgaggaaaagaaatcaaccgagatttcgtcagtagcggcgagcgaaagcgaaagagccagtaagtgatagcagtatagtgaggagaatgtgttgggaagcacaatcaaagagggtgataatcccgtatctaaaaaccatattgtggtactaagctaacgagaagtagggcgggacacgtgatatcctgtttgaagaaggggggaccatcctccaaggctaaatactcctgattgaccgatagtgaaccagtactgtgaaggaaaggcgaaaagaaccccggtgaggggagtgaaatagaacctgaaaccttgtacgtacaagcagtgggagccccatcacttaatccgattggaaggtgatgaaaagcaaccgcacttaaacaatggtaagaggagcgaagcgagtcaaaccacccaaaagaagcagtgggattaagtgatggggtgactgcgtaccttttgtataatgggtcagcgacttatattttgtagcgaggttaaccgaataggggagccgaagggaaaccgagtcttaactgggcgaatagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaatctatttaggtagagccttgaggtgacacctttgggggtagagcactgtttcggctaggggtccatcccggattaccaacccgatgcaaactacgaataccaaagagtgatactcaggagacacacggcgggtgctaacgtccgtcgtggagagggaaacaa

SEQ ID NO: 41 - DNA, 1024 bp, Haemophilus influenzae
ctatctgcgaaaagcttggatgagtcgataagaggcgtttaatccaagatatccgaatggggaaacccagtagatgaagaatctactatcaacaagtgaattcatagcttgttgaggcaaaccgggagaactgaaacatctaagtaccccgaggaaaagaaatcaaccgagatttcgtcagtagcggcgagcgaaagcgaaagagccagtaagtgatagcagtatagtgaggagaatgtgttgggaagcacaatcaaagagggtgataatcccgtatctaaaaaccatattgtggtactaagctaacgagaagtagggcgggacacgtgatatcctgtttgaagaaggggggcccatcctccaaggctaaatactcctgattgaccgatagtgaaccagtactgtgaaggaaaggcgaaaagaaccccggtgaggggagtgaaatagaacctgaaaccttgtacgtacaagcagtgggagccccatcacttaatccgattggaaggtgatgaaaagcaaccgcacttaaacaatggtaagaggagcgaagcgagtcaaaccacccaaaagaagcagtgggattaagtgatggggtgactgcgtaccttttgtataatgggtcagcgacttatattttgtagcgaggttaactgaataagggagccgaagggaaaccgagtcttaactgggcgaagagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaatctatttaggtagagccttgagcttacacctttgggggtagagcactgtttcggctaggggtccatcccggattaccaacccgatgcaaactacgaataccaaagagtgatactcaggagacacacggcgggtgctaacgtccgtcgtggagagggaaacaa

SEQ ID NO: 42 - DNA, 1024 bp, Haemophilus influenzae
ctatctgcgaaaagcttgggtgagtcgataagaggcgtttaatccaagatatccgaatggggaaacccagtagatgaagaatctactatcacttactgaatccataggtaagtgaggcaaaccgggagaactgaaacatctaagtaccccgaggaaaagaaatcaaccgagatttcgtcagtagcggcgagcgaaagcgaaagagcctgttaatgataacagcagagatagaggaataagctgggaagcttagcgacacagggtgatagccccgtactcgacatccaggctgtggtactaagttaacgagaagtagggcgggacacgtgatatcctgtttgaagaaggggggaccatcctccaaggctaaatactcctgattgaccgatagtgaaccagtactgtgaaggaaaggcgaaaagaaccccggtgaggggagtgaaatagaacctgaaaccttgtacgtacaagcagtgggagcctgaaagggtgactgcgtaccttttgtataatgggtcagcgacttatattttgtagcgaggttaaccgaataggggagccaaagggaaaccgagtcttaactgggcgactagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaatctatttaggtagagccttgagcggacacctttgggggtagagcactgtttcggctaggggtccatcccggattaccaacccgatgcaaactacgaataccaaagagtgatactcaggagacacacggcgggtgctaacgtccgtcgtggagagggaaacaacccagaccgccagctaaggtcccaaagtctatattaagtgggaaacgaagtgggaaggcttagacagctaggatgttggcttagaagcagccatcatttaaaga

SEQ ID NO: 43 - DNA, 1024 bp, Haemophilus influenzae
ctatctgcgaaaagcttggatgagtcgataagaggcgtttaatccgagatatccgaatggggaaacccagtagatgaagaatctactatcaacaagtgaattcatagcttgttgaggcaaaccgggagaactgaaacatctaagtaccccgaggaaaagaaatcaaccgagatttcgtcagtagcggcgagcgaaagcgaaagagccagtaagtgatagcagtatagtgaggagaatgtgttgggaagcacaatcaaagagggtgataatcccgtatctaaaaaccatattgtggtactaagctaacgagaagtagggcgggacacgtgatatcctgtttgaagaaggggggaccatcctccaaggctaaatactcctgattgaccgatagtgaaccagtactgtgaaggaaaggcgaaaagaaccccggtgaggggagtgaaatagaacctgaaaccttgtacgtacaagcagtgggagccccatcacttaatccgattggaaggtgatgaaaagcaaccgcacttaaacaatggtaagaggagcgaagcgagtcaaaccacccaaaagaagcagtgggattaagtgatggggtgactgcgtaccttttgtataatgggtcagcgacttatattttgtagcgaggttaaccgaataggggagccgaagggaaaccgagtcttaactgggcgaatagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaatctatttaggtagagccttgaggtgacacctttgggggtagagcactgtttcggctaggggtccatcccggattaccaacccgatgcaaactacgaataccaaagagtgatactcaggagacacacggcgggtgctaacgtccgtcgtggagagggaaacaa

SEQ ID NO: 44 - DNA, 1024 bp, Haemophilus influenzae
aggggctatctgcgaaaagcttggatgagtcgataagaggcgtttaatccaagatatccgaatggggaaacccagtagatgaagaatctactatcaacaagtgaattcatagcttgttgaggcaaaccgggagaactgaaacatctaagtaccccgaggaaaagaaatcaaccgagatttcgtcagtagcggcgagcgaaagcgaaagagccagtaagtgatagcagtatagtgaggagaatgtgttgggaagcacaatcaaagagggtgataatcccgtatctaaaaaccatattgtggtactaagctaacgagaagtagggcgggacacgtgatatcctgtttgaagaaggggggaccatcctccaaggctaaatactcctgattgaccgatagtgaaccagtactgtgaaggaaaggcgaaaagaaccccggtgaggggagtgaaatagaacctgaaaccttgtacgtacaagcagtgggagccccatcacttaatccgattggaaggtgatgaaaagcaaccgcacttaaacaatggtaagaggagcgaagcgagtcaaaccacccaaaagaagcagtgggattaagtgatggggtgactgcgtaccttttgtataatgggtcagcgacttatattttgtagcgaggttaaccgaataggggagccgaagggaaaccgagtcttaactgggcgaatagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaatctatttaggtagagccttgaggtgacacctttgggggtagagcactgtttcggctaggggtccatcccggattaccaacccgatgcaaactacgaataccaaagagtgatactcaggagacacacggcgggtgctaacgtccgtcgtggagaggga

SEQ ID NO: 45 - DNA, 1024 bp, Haemophilus influenzae
ctaatctgcgaaaagcttggatgagtcgataagaggcgtttaatccaagatatccgaatggggaaacccagtagatgaagaatctactatcaacaagtgaattcatagcttgttgaggcaaaccgggagaactgaaacatctaagtaccccgaggaaaagaaatcaaccgagatttcgtcagtagcggcgagcgaaagcgaaagagccagtaagtgatagcagtatagttaggagaatgtgttgggaagcacaatcaaagagggtgataatcccgtatctaaaaaccatattgtggtactaagctaacgagaagtagggcgggacacgtgatatcctgtttgaagaaggggggaccatcctccaaggctaaatactcctgattgaccgatagtgaaccagtactgtgaaggaaaggcgaaaagaaccccggtgaggggagtgaaatagaacctgaaaccttgtacgtacaagcagtgggagccccatcacttaatccgattggaaggtgatgaaaagcaaccgcacttaaacaatggtaagaggagcgaagcgagtcaaaccacccaaaagaagcagtgggattaagtgatggggtgactgcgtaccttttgtataatgggtcagcgacttatattttgtagcgaggttaaccgaataggggagccgaagggaaaccgagtcttaactgggcgagtagttgcaaggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaatctatttaggtagagccttgaggtgacacctttgggggtagagcactgtttcggctagggggccatcccggcttaccaacccgatgcaaactacgaataccaaagagtgatactcaggagacacacggcgggtgctaacgtccgtcgtggagagggaaaca

SEQ ID NO: 46 - DNA, 1024 bp, Mycoplasma pneumoniae
ttacctgcgatagcttcgggtaggtggtaagaacctcagatccggagatttccgaatggagcaatccggtagttggaaacagctatcattaattgatgaataaatagtcatattaaagcaatacgtggtgaagtgaaacatctcagtagccacaggaaaagaaaacgaatgtgattccgtgtgtagtggcgagcgaaagcggaacaggccaaacttatcattagataggggttgtagggcttgcaatgtggacttgaaaacgatagaagaagctgttggaaagcagcgcgcaaaagggtgatagccccgtatttgaaattgttttcatacctagcgagatccctgagtagctcggaaaacgttattttgagtgaatctgcccagaccattgggtaagcctaaatactaattagtgaccgatagcgaaacagtaccgtgagggaaaggtgaaaagaacccagagatgggagtgaaatagattctgaaaccatatgcctacaacgtgtcagagcacattaatgtgtgatggcgtgcgttttgaagtatgagccggcgagttatgatagcaagcgttagttaaccaggagatggggagctgtagcgaaagcgagttttaaaagagcgtttgtttgttattatagacccgaaacgggttgagctagtcatgagcaggttgaaggttgagtaacatcaactggaggaccgaaccgactctcgttgaaacgatagcggatgacttgtgattaggggtgaaattccaatcgaaatccgtgatagctggttctcgtcgaaatagctttaaggctagcgtgagatcacaaataagtggaggtaaagctactgaatgtatgatggcgccacctaggcgtactgaatacaattaaactctgaatgccatttattttattctcgcagtcagacagtgggggataagcttcattgtcaagaggggaagagcccagatcattaaataaggtccccaaaatatactaagtggaaaaggatgtgaaagtgctaaaacagc

SEQ ID NO: 47 - DNA, 1024 bp, Klebsiella pneumoniae
ctatctgcgaaaagcgtcggtaggtgatatgaaccgttataaccggcgatgtccgaatggggaaacccagtgcaattcgttgcactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggttgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccttcgggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatcttcgggggtagagcactgtttcggctagggggtcatcccgacttaccaacccgatgcaaactacgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaaaga

SEQ ID NO: 48 - DNA, 1024 bp, Klebsiella pneumoniae
ctatctgcgaaaagcgtcggtaggtgatatgaaccgttataaccggcgatgtccgaatggggaaacccagtgcaattcgttgcactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggttgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccttcgggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggtcatcccgacttaccaacccgatgcaaactacgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaaaga

SEQ ID NO: 49 - DNA, 1024 bp, Klebsiella pneumoniae; identical to SEQ ID NO: 48

SEQ ID NO: 50 - DNA, 1024 bp, Klebsiella pneumoniae; identical to SEQ ID NO: 48

SEQ ID NO: 51 - DNA, 1024 bp, Klebsiella pneumoniae; identical to SEQ ID NO: 47

SEQ ID NO: 52 - DNA, 1024 bp, Klebsiella pneumoniae
ctatctgcgaaaagcgtcggtaggtgatatgaaccgttacaaccggcgatgtccgaatggggaaacccagtgcaattcgttgcactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggctgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccttcgggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggtcatcccgacttaccaacccgatgcaaactacgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaaaga

SEQ ID NO: 53 - DNA, 1024 bp, Klebsiella pneumoniae
ctatctgcgaaaagcgtcggtaggtgatatgaaccgttataaccggcgatgtccgaatggggaaacccagtgcaattcgttgcactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggctgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccttcgggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggtcatcccgacttaccaacccgatgcaaactacgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaaaga

SEQ ID NO: 54 - DNA, 1024 bp, Klebsiella pneumoniae; identical to SEQ ID NO: 47

SEQ ID NO: 55 - DNA, 1024 bp, Klebsiella pneumoniae
ggttaagcgactaagcgtacacggtggatgccctggcagtcagaggcgatgaaggacgtgctaatctgcgaaaagcgtcggtaaggtgatatgaaccgttataaccggcgatgtccgaatggggaaacccagtgcaattcgttgcactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggctgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccttcgggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggtcatcccgacttaccaacccgatgcaaactacgaataccgaagaatgttatcacgggagacacacggcgggtgctaacgtccgtcgtgaagagggaaacaacccagaccgccagctaaggtcccaaagtcatggttaagtggg

SEQ ID NO: 56 - DNA, 1024 bp, Klebsiella pneumoniae
  misc_feature (56): n is a, c, g, or t
gctaatctgcgaaaagcgtcggtaaggtgatatgaaccgttataaccggcgatgtccgaatggggaaacccagtgtgattcgtcgcactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggttgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccttcgggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaantcatcttcgggggtagagcactgtttcggctagggggtcatcccgacttaccaacccgatgcaaactacgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaa

SEQ ID NO: 57 - DNA, 1024 bp, Klebsiella pneumoniae
  misc_feature (37): n is a, c, g, or t
tctgcgaaagcgtcggtaaggtgatatgaaccgttanagccggcgatgtccgaatggggaaacccagtgtgattcgtcacactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggctgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggncacttcgtggtgtgactgcgntaccttttgtataatgggtcagcgacttatattctgtagcaaggttaaccgtataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggccatcccggcttaccaacccgatgcaaactacgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaaagaaag

SEQ ID NO: 58 - DNA, 1024 bp, Klebsiella pneumoniae; identical to SEQ ID NO: 55

SEQ ID NO: 59 - DNA, 1024 bp, Klebsiella pneumoniae
  misc_feature (288): n is a, c, g, or t
gctaatctgcgaaaagcgccggcgaggtgatatgaacctttgacccggcgatgtccgaatggggaaacccagtgtgattcgtcacactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaagcgtctggaaagtcgcacggtacagggtgacagtcccgtacacgaaanngcacaggttgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaanagaacctgaaaccgtgtacgtacaagcagtgggagcaccttcgtggtgtgactgcgtaccttttgtataatgggtcagcgacttatattctgtagcaaggttaaccgtataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggccatcccggcttaccaacccgatgcaaactacgaataccgaanaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaa

SEQ ID NO: 60 - DNA, 1024 bp, Klebsiella pneumoniae
  misc_feature (42): n is a, c, g, or t
ctaatctgcgaaaagcgtcggtaaggtgatatgaaccgttanagccggcgatgtccgaatggggaaacccagtgtgattcgtcncactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggctgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccttcgggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatcttcgggggtagagcactgtttcggctagggggtcatcccgacttaccaacccgatgcaaactacgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttagaagcagccatcatttaaa

SEQ ID NO: 61 - DNA, 1024 bp, Klebsiella pneumoniae
  misc_feature (297): n is a, c, g, or t
ctaatctgcgaaaagcgtcggtaaggtgatatgaaccgttataaccggcgatgtccgaatggggaaacccagtgcaattcgttgcactatcgttaactgaatacataggttaacgaggcgaaccgggggaactgaaacatctaagtaccccgaggaaaagaaatcaaccgagattcccccagtagcggcgagcgaacggggagcagcccagagtctgaatcagcttgtgtgttagtggaacggtctggaaagtccgacggtacagggtgatagtcccgtacaccaaaatgcacaggntgtgaactcgaagagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccttcgggtgtgactgcgtaccttttgtataatgggtcagcgacttatattctgtagcaaggttaaccgtataggggagccgcagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaactcatcttcgggggtagagcactgtttcggctagggggtcatcccgacttaccaacccgatgcaaactacgaataccgaagaatgttatcacgggagacacacggcgggtgctaacgtccgtcgtgaagagggaaacaacccagaccgccagctaaggtcccaaagtcatggttaagtgggaaacgatgtgggaaggcacagacagccaggatgttggcttanaancagccatcatttaaa

SEQ ID NO: 62 - DNA, 1024 bp, Legionella pneumophila
ggaacgagcgttgagccatagatgtccgaatgggggaacccggctgcagcgatgcggtcatttgcatttgaatacataggatgcaaaggcgaactcggggaactgaaacatctaagtacccgaaggaaaagaaatcaagagagattctccaagtagcggcgagcgaacggggaggagcctggcgtgatttattattgaactgagtagaacaatttgggaaagttggccgtagagggtgaaagccccgtatacgaaggtttgatgaggaactaggcacgcgaacaagtaggccgggacacgtgaaatcctggttgaagatgggtggaccatcatccaaggctaaatactacttactgaccgatagtgaaccagtaccgtgagggaaaggtgaaaagaaccccggagaggggagtgaaatagaatctgaaaccgtttgcgtacaagcagtgggagcatagtttaggctgtgtgactgcgtaccttttgtataatgggtcagcgagttactttcagtggcgaggttaactgaataagggagccgtagagaaatcgagtctgaatagggcgatagtcgctgggagtagacccgaaaccgggcgatctagccatgtgcaggatgaaggttgggtaacaccaactggaggtccgaaccgggtaatgttgaaaaattatcggatgacgtgtggctaggagtgaaaggctaatcaagcccggagatagctggttctccccgaaagctatttaggtagcgcctcgtgaatgattactgggggtagagcactgtttcggctagggggctgtcatggcttaccaaaccgatgcaaactccgaataccggctaattgaatcacgggagacacacggcgggtgctaacgtccgtcgtggagagggaaacaacccagaccgccagctaaggtccccaagtactagttaagtgggaaacgatgtgggaaggcatagacagccaggaggttggcttagaagcagccaccctttaaagaaagcgtaatagctcactggtcg

SEQ ID NO: 63 - DNA, 1024 bp, Legionella pneumophila; identical to SEQ ID NO: 62

SEQ ID NO: 64 - DNA, 1024 bp, Legionella pneumophila; identical to SEQ ID NO: 62

SEQ ID NO: 65 - DNA, 1024 bp, Moraxella catarrhalis
gcgatagcgtcggtgaggtggcaatatcctgtgacccggcgatttctgaatggggaaacccaaccaacataagttggttattacacagtttactgtgtaaggcaaaccgggagaagtgaaacatctcagtaccccgaggaaaagacatcaaatgagattccgtaagtagcggcgagcgaacacggaggagccgatcaattttacagtagcaaaatggcgtgggaaagccaaccatagtaggtgatagtcctgtatgcgaaactgtttaagcgacatattaagtagggcggaacacgagaaattctgtctgaagatggggggaccatcctccaaggctaaatactcctgactgaccgatagtgaaccagtaccgtgagggaaaggcgaaaagaacccctgttaggggagtgaaatagaacctgaaaccgtgtgcatacaagcagtcggagcccgaccacttaatcgttttgaggataacaatgaatttttactacgtctatgtcatacaaaacgtggataatcatgatgagttttatgttggttttaccaccaatttaaaacaaagaattgatgaccataataatggcacaacttactcaaccagagcaagaacatggcgtattgtttattgtgaagcctacattaatgaacaagtggcaagaaaacgagagcgaaccataaaaaataatggtcgtatgagaactttcttgatgaatcgtgtcaaatcacaatttgacaatcagaacgattaagtggtcgggtgacggcgtaccttttgtataatgggtcagcgacttatattctgtagcaaggttaaccgaataggggagccgtagggaaaccgagtcttaatagggcgattaagttgcagggtatagacccgaaaccgagtgatctatccatgagcaggttgaaagtgccgtaacaggcaccggaggaccgaacccactgtcgttgaaaagccaggggatgacttgtggataggggtgaaaggctaatcaaactcggtgatagctggttctcc

SEQ ID NO: 66 - DNA, 1024 bp, Moraxella catarrhalis
gcgatagcgtcggtgaggtggcaatatcctgtgacccggcgatttctgaatggggaaacccaaccaacataagttggttattacacagtttactgtgtaaggcaaaccgggagaagtgaaacatctcagtaccccgaggaaaagacatcaaatgagattccgtaagtagcggcgagcgaacacggaggagccgatcaattttacagtagcaaaatggcgtgggaaagccaaccatagtaggtgatagtcctgtatgcgaaactgtttaagcgacatattaagtagggcggaacacgagaaattctgtctgaagatggggggaccatcctccaaggctaaatactcctgactgaccgatagtgaaccagtaccgtgagggaaaggcgaaaagaacccctgttaggggagtgaaatagaacctgaaaccgtgtgcatacaagcagtcggagcccgaccacttaatcgttttgaggataacaatgaatttttactacgtctatgtcatacaaaacgtggataatcatgatgagttttatgttggctttaccaccaatttaaaacaaagaattgatgaccataataatggcacaacttactcaaccagagcaagaacatggcgtattgtttattgtgaagcctacattaatgaacaagtggcaagaaaacgagagcgaaccataaaaaataatggtcgtatgagaactttcttgatgaatcgtgtcaaatcacaatttgacaatcagaacgattaagtggtcgggtgacggcgtaccttttgtataatgggtcagcgacttatattctgtagcaaggttaaccgaataggggagccgtagggaaaccgagtcttaatagggcgattaagttgcagggtatagacccgaaaccgagtgatctatccatgagcaggttgaaagtgccgtaacaggcaccggaggaccgaacccactgtcgttgaaaagccaggggatgacttgtggataggggtgaaaggctaatcaaactcggtgatagctggttctcc

SEQ ID NO: 67 - DNA, 1024 bp, Moraxella catarrhalis
gcgatagcgtcggtgaggtggcaatatcctgtgacccggcgatttctgaatggggaaacccaaccaacataagttggttattacacagtttactgtgtaaggcaaaccgggagaagtgaaacatctcagtaccccgaggaaaagacatcaaatgagattccgtaagtagcggcgagcgaacacggaggagccgatcaattttacagtagcaaaatggcgtgggaaagccaaccatagtaggtgatagtcctgtatgcgaaactgtttaagcgacatattaagtagggcggaacacgagaaattctgtctgaagatggggggaccatcctccaaggctaaatactcctgactgaccgatagtgaaccagtaccgtgagggaaaggcgaaaagaacccctgttaggggagtgaaatagaacctgaaaccgtgtgcatacaagcagtcggagcccgaccacttaatcgttttgaggataacaatgaatttttactacgtctatgtcatacaaaacgtggataatcatgatgagttttatgttggttttaccaccaatttaaaacaaagaattgatgaccataataatggcacaacttactcaaccagagcaagaacatggcgtattgtttattgtgaagcctacattaatgaacaagtggcaagaaaacgagagcgaaccataaaaaataatggtcgtatgagaactttcttgatgaatcgtgtcaaatcacaatttgacaatcagaacgattaagtggtcgggtgacggcgtaccttttgtataatgggtcagcgacttatattctgtagcaaggttaaccgaataggggagccgtagggaaaccgagtcttaatagggcgaatgagttgcagggtatagacccgaaaccgagtgatctatccatgagcaggttgaaagtgccgtaacaggcaccggaggaccgaacccactgtcgttgaaaagccaggggatgacttgtggataggggtgaaaggctaatcaaactcggtgatagctggttctcc

SEQ ID NO: 68 - DNA, 1024 bp, Pseudomonas aeruginosa
cctgcgaaagcttcggggagtcggcaaacagactttgatccggagatctctgaatgggggaacccacctaggataacctaggtatcttgtactgaatccataggtgcaagaggcgaaccaggggaactgaaacatctaagtaccctgaggaaaagaaatcaaccgagattcccttagtagtggcgagcgaacggggattagcccttaagcttcattgattttagcggaacgctctggaaagtgcggccatagtgggtgatagccccgtacgcgaaaggatctttgaagtgaaatcgagtaggacggagcacgagaaactttgtctgaacatggggggaccatcctccaaggctaaatactactgactgaccgatagtgaaccagtaccgtgagggaaaggcgaaaagaaccccggagaggggagtgaaatagaacctgaaaccgtatgcgtacaagcagtgggagcctacttgttaggtgactgcgtaccttttgtataatgggtcagcgacttatattcagtggcaagcttaaccgtatagggtaggcgtagcgaaagcgagtcttaatagggcgtttagtcgctgggtatagacccgaaaccgggcgatctatccatgagcaggttgaaggttaggtaacactgactggaggaccgaacccactcccgttgaaaaggtaggggatgacttgtggatcggagtgaaaggctaatcaagctcggagatagctggttctcctcgaaagctatttaggtagcgcctcatgtatcactctggggggtagagcactgtttcggctagggggtcatcccgacttaccaaaccgatgcaaactccgaatacccagaagtgccgagcatgggagacacacggcgggtgctaacgtccgtcgtgaaaagggaaacaacccagaccgccagctaaggtcccaaagttgtggttaagtggtaaacgatgtgggaaggcttagacagctaggaggttggcttagaagcagccatcctttaaagaaagcgtaatag

SEQ ID NO: 69 - DNA, 1024 bp, Pseudomonas aeruginosa
cctgcgaaagcttcggggagtcggcaaacagactttgatccggagatctctgaatgggggaacccacctaggataacctaggtatcttgtactgaatccataggtgcaagaggcgaaccaggggaactgaaacatctaagtaccctgaggaaaagaaatcaaccgagattcccttagtagtggcgagcgaacggggattagcccttaagcttcattgattttagcggaacgctctggaaagtgcggccatagtgggtgatagccccgtacgcgaaaggatctttgaagtgaaatcgagtaggacggagcacgagaaactttgtctgaacatggggggaccatcctccaaggctaaatactactgactgaccgatagtgaaccagtaccgtgagggaaaggcgaaaagaaccccggagaggggagtgaaatagaacctgaaaccgtatgcgtacaagcagtgggagcctacttgttaggtgactgcgtaccttttgtataatgggtcagcgacttatattcagtggcaagcttaaccgtatagggtaggcgtagcgaaagcgagtcttaatagggcgtttagtcgctgggtatagacccgaaaccgggcgatctatccatgagcaggttgaaggttaggtaacactgactggaggaccgaacccactcccgttgaaaaggtaggggatgacttgtggatcggagtgaaaggctaatcaagctcggagatagctggttctcctcgaaagctatttaggtagcgcctcatgtatcactctggggggtagagcactgtttcggctagggggtcatcccgacttaccaaaccgatgcaaactccgaatacccagaagtgccgagcatgggagacacacggcgggtgctaacgtccgtcgtgaaaagggaaacaacccagaccgccagctaaggtcccaaagttgtggttaagtggtaaacgatgtgggaaggcttagacagctaggaggttggcttagaagcagccaccctttaaagaaagcgtaatag

SEQ ID NO: 70 - DNA, 1024 bp, Pseudomonas aeruginosa; identical to SEQ ID NO: 69

SEQ ID NO: 71 - DNA, 1024 bp, Pseudomonas aeruginosa; identical to SEQ ID NO: 68

SEQ ID NO: 72 - DNA, 1024 bp, Pseudomonas aeruginosa; identical to SEQ ID NO: 68

SEQ ID NO: 73 - DNA, 1024 bp, Pseudomonas aeruginosa; identical to SEQ ID NO: 68

SEQ ID NO: 74 - DNA, 1024 bp, Pseudomonas aeruginosa; identical to SEQ ID NO: 68

SEQ ID NO: 75 - DNA, 1024 bp, Pseudomonas aeruginosa; identical to SEQ ID NO: 68

SEQ ID NO: 76 - DNA, 1024 bp, Pseudomonas aeruginosa
ggtcaagtgaagaagcgcatacggtggatgccttggcagtcagaggcgatgaaagacgtggtagcctgcgaaaagcttcggggagtcggcaaacagactttgatccggagatctctgaatgggggaacccacctaggataacctaggtatcttgtactgaatccataggtgcaagaggcgaaccacggggaactgaaacatctaagtaccctgaggaaaagaaatcaaccgagattcccttagtagtggcgagcgaacggggattagcccttaagcttcattgattttagcggaacgctctggaaagtgcggccatagtgggtgatagccccgtacgcgaaaggatctttgaagtgaaatcgagtaggacggagcacgagaaactttgtctgaacatggggggaccatcctccaaggctaaatactactgactgaccgatagtgaaccagtaccgtgagggaaaggcgaaaagaaccccggagaggggagtgaaatagaacctgaaaccgtatgcgtacaagcagtgggagcctacttgttaggtgactgcgtaccttttgtataatgggtcagcgacttatattcagtggcaagcttaatcgtatagggtaggcgtagcgaaaagcgagtcttaatagggcgtttagtcgctgggtatagacccgaaaccgggcgatctatccatgagcaggttgaaggttaggtaacactgactggaggaccgaacccactcccgttgaaaaggtaggggatgacttgtggatcggagtgaaaggctaatcaagctcggagatagctggttctcctcgaaagctatttaggtagcgcctcatgtatcactctggggggtagagcaactgtttcggctagggggtcatcccgacttaccaaaccgatgcaaactccgaatacccagaagtgccgagcatgggagacacacggcgggtgctaacgtccgtcgtgaaaagggaaacaacccagaccgccagctaaggtcccaaagttgtggttaagtggtaaacg

SEQ ID NO: 77 - DNA, 1024 bp, Staphylococcus aureus
tgtaagtaagctttgatccagagatttccgaatggggaaacccagcatgagttatgtcatgttatcgatatgtgaatacatagcatatcagaaggcacacccggagaactgaaacatcttagtacccggaggaagagaaagaaaattcgattcccttagtagcggcgagcgaaacgggaagagcccaaaccaacaagcttgcttgttggggttgtaggacactctatacggagttacaaaggacgacattagacgaatcatctggaaagatgaatcaaagaaggtaataatcctgtagtcgaaaatgttgtctctcttgagtggatcctgagtacgacggagcacgtgaaattccgtcggaatctgggaggaccatctcctaaggctaaatactctctagtgaccgatagtgaaccagtaccgtgagggaaaggtgaaaagcaccccggaaggggagtgaaatagaacctgaaaccgtgtgcttacaagtagtcagagcccgttaatgggtgatggcgtgccttttgtagaatgaaccggcgagttacgatttgatgcaaggttaagcagtaaatgtggagccgtagcgaaagcgagtctgaatagggcgtttagtatttggtcgtagacccgaaaccaggtgatctacccttggtcaggttgaagttcaggtaacactgaatggaggaccgaaccgacttacgttgaaaagtgagcggatgaactgagggtagcggagaaattccaatcgaacctggagatagctggttctctccgaaatagctttagggctagcctcaagtgatgattattggaggtagagcactgtttggacgaggggcccctctcgggttaccgaattcagacaaactccgaatgccaattaatttaacttgggagtcagaacatgggtgataaggtccgtgttcgaaagggaaacagcccagaccaccagctaaggtcccaaaatatatgttaagtggaaaaggatgtggcgttgcccagacaactaggatgttggctt

SEQ ID NO: 78 - DNA, 1024 bp, Staphylococcus aureus; identical to SEQ ID NO: 77

SEQ ID NO: 79 - DNA, 1024 bp, Staphylococcus aureus; identical to SEQ ID NO: 77

SEQ ID NO: 80 - DNA, 1024 bp, Staphylococcus aureus
gattaagttattaagggcgcacggtggatgccttggcactagaagccgatgaaggacgttactaacgacgatatgctttggggagctgtaagtaagctttgatccagagatttccgaatggggaaacccagcatgagttatgtcatgttatcgatatgtgaatacatagcatatcagaaggcacacccggagaactgaaacatcttagtacccggaggaagagaaagaaaattcgattcccttagtagcggcgagcgaaacgggaagagcccaaaccaacaagcttgcttgttggggttgtaggacactctatacggagttacaaaggacgacattagacgaatcatctggaaagatgaatcaaagaaggtaataatcctgtagtcgaaaatgttgtctctcttgagtggatcctgagtacgacggagcacgtgaaattccgtcggaatctgggaggaccatctcctaaggctaaatactctctagtgaccgatagtgaaccagtaccgtgagggaaaggtgaaaagcaccccggaaggggagtgaaatagaacctgaaaccgtgtgcttacaagtagtcagagcccgttaatgggtgatggcgtgccttttgtagaatgaaccggcgagttacgatttgatgcaaggttaagcagtaaatgtggagccgtagcgaaagcgagtctgaatagggcgtttagtatttggtcgtagacccgaaaccaggtgatctacccttggtcaggttgaagttcaggtaacactgaatggaggaccgaaccgacttacgttgaaaagtgagcggatgaactgagggtagcggagaaattccaatcgaacctggagatagctggttctctccgaaatagctttagggctagcctcaagtgatgattattggaggtagagcactgtttggacgaggggcccctctcgggttaccgaattcagacaaactccgaatgccaattaatttaacttgggagtcagaacatgggtgataaggtccgtgttcgaaagggaa

SEQ ID NO: 81 - DNA, 1024 bp, Staphylococcus aureus
  misc_feature (47): n is a, c, g, or t
cgacgatatgctttggggagctgtaagtaagctttgatccagagatntccgaatggggaaacccagcatgagttatgtcatgttatcgatatgtgaatacatagcatatcagaaggcacacccggagaactgaaacatcttagtacccggaggaagagaaagaaaattcgattcccttagtagcggcgagcgaaacgggaagagcccaaaccaacaagcttgcttgttggggttgtaggacactctatacggagttacaaaggacgacattagacgaatcatctggaaagatgaatcaaagaaggtaataatcctgtagtcgaaaatgttgtctctcttgagtggatcctgagtacgacggagcacgtgaaattccgtcggaatctgggaggaccatctcctaaggctaaatactctctagtgaccgatagtgaaccagtaccgtgagggaaaggtgaaaagcaccccggaaggggagtgaaatagaacctgaaaccgtgtgcttacaagtagtcagagcccgttaatgggtgatggcgtgccttttgtagaatgaaccggcgagttacgatttgatgcaaggttaagcagtaaatgtggagccgtagcgaaagcgagtctgaatagggcgtttagtatttggtcgtagacccgaaaccaggtgatctacccttggtcaggttgaagttcaggtaacactgaatggaggaccgaaccgacttacgttgaaaagtgagcggatgaactgagggtagcggagaaattccaatcgaacctggagatagctggttctctccgaaatagctttagggctagcctcaagtgatgattattggaggtagagcactgtttggacgaggggcccctctcgggttaccgaattcagacaaactccgaatgccaattaatttaacttgggagtcagaacatgggtgataaggtccgtgttcgaaagggaaacagcccagaccaccagctaaggtcccaaaatatatgttaagtggaaaaggatgtggcgttgccc

SEQ ID NO: 82 - DNA, 1024 bp, Staphylococcus aureus
cgacgatatgctttggggagctgtaagtaagctttgatccagagatttccgaatggggaaacccagcatgagttatgtcatgttatcgatatgtgaatacatagcatatcagaaggcacacccggagaactgaaacatcttagtacccggaggaagagaaagaaaattcgattcccttagtagcggcgagcgaaacgggaagagcccaaaccaacaagcttgcttgttggggttgtaggacactctatacggagttacaaaggacgacattagacgaatcatctggaaagatgaatcaaagaaggtaataatcctgtagtcgaaaatgttgtctctcttgagtggatcctgagtacgacggagcacgtgaaattccgtcggaatctgggaggaccatctcctaaggctaaatactctctagtgaccgatagtgaaccagtaccgtgagggaaaggtgaaaagcaccccggaaggggagtgaaatagaacctgaaaccgtgtgcttacaagtagtcagagcccgttaatgggtgatggcgtgccttttgtagaatgaaccggcgagttacgatttgatgcaaggttaagcagtaaatgtggagccgtagcgaaagcgagtctgaatagggcgtttagtatttggtcgtagacccgaaaccaggtgatctacccttggtcaggttgaagttcaggtaacactgaatggaggaccgaaccgacttacgttgaaaagtgagcggatgaactgagggtagcggagaaattccaatcgaacctggagatagctggttctctccgaaatagctttagggctagcctcaagtgatgattattggaggtagagcactgtttggacgaggggcccctctcgggttaccgaattcagacaaactccgaatgccaattaatttaacttgggagtcagaacatgggtgataaggtccgtgttcgaaagggaaacagcccagaccaccagctaaggtcccaaaatatatgttaagtggaaaaggatgtggcgttgccc

SEQ ID NO: 83 - DNA, 1024 bp, Staphylococcus aureus; identical to SEQ ID NO: 77

SEQ ID NO: 84 - DNA, 1024 bp, Streptococcus pneumoniae
ggttaagttaataagggcgcacggtggatgccttggcactaggagccgacgaaggacgtgacaaacgacgatatgccttgggtagctgtaagtaagcgatgatccatggatttccgaatgggggaacccaacaggtaatacctgttacccacatctgttaaggatgtgaggaggaagacgcagtgaactgaaacatctaagtagctgcaggaagagaaagcaaaagcgattgccttagtagcggcgagcgaaacggcagaagggcaaaccgaagagtttactcttcggggttgtaggactgcaatgtggactcaaagattatagaagaatgatttgggaagatcagccaaagagagtaatagcctcgtatttaaaatagtctttgtacctagcagtatcctgagtacggcgggacacgtgaaatcccgtcggaatctgggaggaccatctcccaaccctaaatactccctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaatagggcgccttagtatcatgacgtagacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagac

SEQ ID NO: 85 - DNA, 1024 bp, Streptococcus pneumoniae
cgacgatatgccttgggtagctgtaagtaagcgatgatccagggatttccgaatgggggaacccaacaggtaatacctgttacccacatctgttaaggatgtgaggaggaagacgcagtgaactgaaacatctaagtagctgcaggaagagaaagcaaaagcgattgccttagtagcggcgagcgaaacggcagaagggcaaaccgaagagtttactcttcggggttgtaggactgcaatgtggactcaaagattatagaagaatgatttgggaagatcagccaaagagagtaatagcctcgtatttaaaatagtctttgtacctagcagtatcctgagtacggcgggacacgtgaaatcccgtcggaatctgggaggaccatctcccaaccctaaatactccctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaatagggcgccttagtatcatgacgtagacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagaccaccagctaaggtcccaaaataattgttaagtggaaaaggatgtggggttgcacagacaactagg

SEQ ID NO: 86 - DNA, 1024 bp, Streptococcus pneumoniae
cgacgatatgccttgggtagctgtaagtaagcgatgatccagggatttccgaatgggggaacccaacaggtaatacctgttacccacatctgttaaggatgtgaggaggaagacgcagtgaactgaaacatctaagtagctgcaggaagagaaagcaaaagcgattgccttagtagcggcgagcgaaacggcagaagggcaaaccgaagagtttactcttcggggttgtaggactgcaatgtggactcaaagattatagaagaatgatttgggaagatcagccaaagagagtaatagcctcgtatttaaaatagtctttgtacttagcagtatcctgagtacggcgggacacgtgaaatcccgtcggaatctgggaggaccatctcccaaccctaaatactccctagtgaccgatagtgaaccagtaccgtgagggaaaggtgaaaagcaccccgtgaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaatagggcgccttagtatcatgacgtagacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagaccaccagctaaggtcccaaaataattgttaagtggaaaaggatgtggggttgcacagacaactagg

SEQ ID NO: 87 - DNA, 1024 bp, Streptococcus pneumoniae
  misc_feature (636): n is a, c, g, or t
cgacgatatgccttgggtagctgtaagtaagcgatgatccagggatttccgaatgggggaacccaacaggtaatacctgttacccacatctgttaaggatgtgaggaggaagacgcagtgaactgaaacatctaagtagctgcaggaagagaaagcaaaagcgattgccttagtagcggcgagcgaaacggcagaagggcaaaccgaagagtttactcttcggggttgtaggactgcaatgtggactcaaagattatagaagaatgatttgggaagatcagccaaagagagtaatagcctcgtatttaaaatagtctttgtacctagcagtatcctgagtacggcgggacacgtgaaatcccgtcggaatctgggaggaccatctcccaaccctaaatactccctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaacagggcgaattagtatcatgacgtanacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagaccaccagctaaggtcccaaaataattgttaagtggaaaaggatgtggggttgcacagacaactagg

SEQ ID NO: 88 - DNA, 1024 bp, Streptococcus pneumoniae
cgacgatatgccttgggtagctgtaagtaagcgatgatccagggatttccgaatgggggaacccaacaggtaatacctgttacccacatctgttaaggatgtgaggaggaagacgcagtgaactgaaacatctaagtagctgcaggaagagaaagcaaaagcgattgccttagtagcggcgagcgaaacggcagaagggcaaaccgaagagtttactcttcggggttgtaggactgcaatgtggactcaaagattatagaagaatgatttgggaagatcagccaaagagagtaatagcctcgtatttaaaatagtctttgtacttagcagtatcctgagtacggcgggacacgtgaaatcccgtcggaatctgggaggaccatctcccaaccctaaatactccctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaatagggcgccttagtatcatgacgtagacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagaccaccagctaaggtcccaaaataattgttaagtggaaaaggatgtggggttgcacagacaactagg

SEQ ID NO: 89 - DNA, 1024 bp, Streptococcus pneumoniae; identical to SEQ ID NO: 85

SEQ ID NO: 90 - DNA, 1024 bp, Streptococcus pneumoniae
cgacgatatgccttgggtagctgtaagtaagcgatgatccagggatttccgaatgggggaacccaacaggtaatacctgttacccacatctgttaaggatgtgaggaggaagacgcagtgaactgaaacatctaagtagctgcaggaagagaaagcaaaagcgattgccttagtagcggcgagcgaaacggcaggagggcaaaccgaagagtttactcttcggggttgtaggactgcaatgtggactcaaagattatagaagaatgatttgggaagatcagccaaagagagtaatagcctcgtatttaaaatagtctttgtacttagcagtatcctgagtacggcgggacacgtgaaatcccgtcggaatctgggaggaccatctcccaaccctaaatactccctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaatagggcgccttagtatcatgacgtagacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagaccaccagctaaggtcccaaaataattgttaagtggaaaaggatgtggggttgcacagacaactagg

SEQ ID NO: 91 - DNA, 1024 bp, Streptococcus pneumoniae; identical to SEQ ID NO: 90

SEQ ID NO: 92 - DNA, 1024 bp, Streptococcus pneumoniae
cgacgatatgccttgggtagctgtaagtaagcgatgatccagggatttccgaatgggggaacccaacaggtaatacctgttacctacatctgttaaggatgtgaggaggaagacgcagtgaactgaaacatctaagtagctgcaggaagagaaagcaaaagcgattgccttagtagcggcgagcgaaacggcagaagggcaaaccgaagagtttactcttcggggttgtaggactgcaatgtggactcaaagattatagaagaatgatttgggaagatcagccaaagagagtaatagcctcgtatttaaaatagtctttgtacttagcagtatcctgagtacggcgggacacgtgaaatcccgtcggaatctgggaggaccatctcccaaccctaaatactccctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaatagggcgccttagtatcatgacgtagacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagaccaccagctaaggtcccaaaataattgttaagtggaaaaggatgtggggttgcacagacaactagg

SEQ ID NO: 93 - DNA, 1024 bp, Streptococcus pneumoniae
cgacgatatgccttgggtgctgtaagtaagcgatgatccagggatttccgaatgggggaacccaacaggtagtacctgttacccacatctgttaaggatgtgaggaggaagacgcagtgaactgaaacatctaagtagctgcaggaagagaaagcaaaagcgattgccttagtagcggcgagcgaaacggcaggagggcaaaccgaagagtttactcttcggggttgtaggactgcaatgtggactcaaagattatagaagaatgatttgggaagatcagccaaagagagtaatagcctcgtatttaaaatagtctttgtacttagcagtatcctgagtacggcgggacacgtgaaatcccgtcggaatctgggaggaccatctcccaaccctaaatactccctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaatagggcgccttagtatcatgacgtagacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagaccaccagctaaggtcccaaaataattgttaagtggaaaaggatgtggggttgcacagacaactagga

SEQ ID NO: 94 - DNA, 1024 bp, Streptococcus pneumoniae
  misc_feature (144): n is a, c, g, or t
cgacgatatgccttgggtgctgtaagtaagcgatgatccagggatttccgaatgggggaacccaacaggtaatacctgtttacccacatctgttaaggatgtgaggaggaagacgcagtgaactgaaacatctaagtagctgcnggaagagaaagcaaaagcgattgccttagtagcggcgagcgaaacggcagaagggcaaaccgaagagtttactcttcggggttgtaggactgcaatgtggactcaaagattatagaagaatgatttgggaagatcagccaaagagagtaatagcctcgtatttaaaatagtctttgtacctagcagtatcctgagtacggcgggacacgtgaaatcccgtcggaatctgggaggaccatctcccaaccctaaatactccctagtgaccgatagtgaaccagtaccgtgagggaaaggtgaaaagcaccccgggaggggagtgaaatagaacctgaaaccgtgtgcctacaacaagttcgagcccgttaatgggtgagagcgtgccttttgtagaatgaaccggcgagttacgttatgatgcgaggttaagttgaagagacggagccgtagggaaaccgagtctgaatagggcgccttagtatcatgacgtagacccgaaaccatgtgacctacccatgagcaggttgaaggtgcggtaagacgcactggaggaccgaaccagggcacgttgaaaagtgcttggatgacttgtgggtagcggagaaattccaaacgaacttggagatagctggttctctccgaaatagctttagggctagcgtcgacattagagattcttggaggtagagcactgtttgggtgaggggtccatcccggattaccaatctcagataaactccgaatgccaatgaattatggtcggcagtcagactgcgagtgctaagatccgtagtcgaaagggaaacagcccagaccaccagctaaggtcccaaaataattgttaagtggaaaaggatgtggggttgcacagacaactagg